SEQUENCE LISTING (40 sequences)

SEQ ID NO: 1 - protein, 876 aa, Clostridioides difficile
MKVQMRNKKVLSFLTLTAIVSQALAYPVYAQTSTSSHSDNKKEIINEDILTNNGLMGYYFTDEHFKDLKLMAPIKDGNLKFEEKKVDKLLNKDKSNVKSIRWTGRIIPSKDGEYTLSTDRDDILMQVNNESTISNTLKVNMKKGKEYKFRIELQDKNLGSIDNLSSPNLYWELDGIKKIIPAENLFLRDYSNIEKNDPFIPNNNFFDPRLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQNPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNQDGLQGSANKLNGETKIKIPMSKLKPYKRYVFSGYSKDPLTSNSIIVKIKAKEEKTDYLVPEQGYTKFSYEFETTEKDSSNIEITLIGSGTTYLDNLSITELNSTPEILNEPEVKIPTDQEIIDAHKIYSADLNFNPSTGNAYINGMYFTPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 2 - protein, 876 aa, Clostridioides difficile; identical to SEQ ID NO: 1

SEQ ID NO: 3 - protein, 876 aa, Clostridioides difficile; identical to SEQ ID NO: 1

SEQ ID NO: 4 - protein, 876 aa, Clostridioides difficile; identical to SEQ ID NO: 1

SEQ ID NO: 5 - protein, 876 aa, Clostridioides difficile; identical to SEQ ID NO: 1

SEQ ID NO: 6 - protein, 876 aa, Clostridioides difficile
MKVQMRNKKVLSFLTFTAIVSQALVYPVYAQTSTSNHSNKKKEIINEDILPNNGLMGYYFTDEHFKDLKLMAPIKDGNLKFEEKKVDKLLDKDKSDVKSIRWTGRIIPSKDGEYTLSTDRDDVLMQVNTESTISNKLKVNMKKGKEYKVRIELQDKNLGSIDNLSSPNLYWELDGIKKIIPEENLFLRDYSNIEKNDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPMALNTMDQFSSRLIPINYDQLKKLDAGKQIKLETTQVSGNFGTKNSSGQIVTEGNSWSDYISQIDSISASIILDTENESYERRVTAKNFQDPEDKTPELTIGEAIEKAFGATKKDGLLYFNDIPIDESCVELIFDDNTANKIKDSLKTLSDKQIYNVKLERGMNILIKTPTYFINFDDYNNYPSIWSNVNTTNQDGLQGSANKLNGETKIKIPMSELKPYKRYVFSGYSKDPLTSNSIIVKIKAKEEKTDYLVPEQEYTKFSYEFETTEKDSSNIEITLIGSGTTYLDNLSITELNSTPEILDEPEVKIPTDQEIIDAHKIYSSDLNFNPSTGNAYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 7 - protein, 876 aa, Clostridioides difficile
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSTIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 8 - protein, 876 aa, Clostridioides difficile
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSNIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 9 - protein, 876 aa, Clostridioides difficile
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSNIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNQDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIMDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 10 - protein, 876 aa, Clostridioides difficile; identical to SEQ ID NO: 9

SEQ ID NO: 11 - protein, 130 aa, Clostridioides difficile
PTYFTNFDDYNNYPSTWSNVNTTNQDGLQGSANKLNGETKIKIPMSKLKPYKRYVFSGYSKDPLTSNSIIVKIKAKEEKTDYLVPEQGYTKFSYEFETTEKDSSNIEITLIGSGTTYLDNLSITELNSTP

SEQ ID NO: 12 - protein, 130 aa, Clostridioides difficile; identical to SEQ ID NO: 11

SEQ ID NO: 13 - protein, 130 aa, Clostridioides difficile; identical to SEQ ID NO: 11

SEQ ID NO: 14 - protein, 130 aa, Clostridioides difficile; identical to SEQ ID NO: 11

SEQ ID NO: 15 - protein, 130 aa, Clostridioides difficile; identical to SEQ ID NO: 11

SEQ ID NO: 16 - protein, 130 aa, Clostridioides difficile
PTYFINFDDYNNYPSIWSNVNTTNQDGLQGSANKLNGETKIKIPMSELKPYKRYVFSGYSKDPLTSNSIIVKIKAKEEKTDYLVPEQEYTKFSYEFETTEKDSSNIEITLIGSGTTYLDNLSITELNSTP

SEQ ID NO: 17 - protein, 130 aa, Clostridioides difficile
PTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTP

SEQ ID NO: 18 - protein, 130 aa, Clostridioides difficile; identical to SEQ ID NO: 17

SEQ ID NO: 19 - protein, 130 aa, Clostridioides difficile
PTYFTNFDDYNNYPSTWSNVNTTNQDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTP

SEQ ID NO: 20 - protein, 130 aa, Clostridioides difficile; identical to SEQ ID NO: 19

SEQ ID NO: 21 - protein, 120 aa, Clostridioides difficile
TDQEIIDAHKIYSADLNFNPSTGNAYINGMYFTPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 22 - protein, 120 aa, Clostridioides difficile; identical to SEQ ID NO: 21

SEQ ID NO: 23 - protein, 120 aa, Clostridioides difficile; identical to SEQ ID NO: 21

SEQ ID NO: 24 - protein, 120 aa, Clostridioides difficile; identical to SEQ ID NO: 21

SEQ ID NO: 25 - protein, 120 aa, Clostridioides difficile; identical to SEQ ID NO: 21

SEQ ID NO: 26 - protein, 120 aa, Clostridioides difficile
TDQEIIDAHKIYSSDLNFNPSTGNAYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 27 - protein, 120 aa, Clostridioides difficile
TDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 28 - protein, 120 aa, Clostridioides difficile; identical to SEQ ID NO: 27

SEQ ID NO: 29 - protein, 120 aa, Clostridioides difficile
TDQEIMDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 30 - protein, 120 aa, Clostridioides difficile; identical to SEQ ID NO: 29

SEQ ID NO: 31 - protein, 261 aa, Clostridioides difficile
PTYFTNFDDYNNYPSTWSNVNTTNQDGLQGSANKLNGETKIKIPMSKLKPYKRYVFSGYSKDPLTSNSIIVKIKAKEEKTDYLVPEQGYTKFSYEFETTEKDSSNIEITLIGSGTTYLDNLSITELNSTPEILNEPEVKIPTDQEIIDAHKIYSADLNFNPSTGNAYINGMYFTPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 32 - protein, 261 aa, Clostridioides difficile; identical to SEQ ID NO: 31

SEQ ID NO: 33 - protein, 261 aa, Clostridioides difficile; identical to SEQ ID NO: 31

SEQ ID NO: 34 - protein, 261 aa, Clostridioides difficile; identical to SEQ ID NO: 31

SEQ ID NO: 35 - protein, 261 aa, Clostridioides difficile; identical to SEQ ID NO: 31

SEQ ID NO: 36 - protein, 261 aa, Clostridioides difficile
PTYFINFDDYNNYPSIWSNVNTTNQDGLQGSANKLNGETKIKIPMSELKPYKRYVFSGYSKDPLTSNSIIVKIKAKEEKTDYLVPEQEYTKFSYEFETTEKDSSNIEITLIGSGTTYLDNLSITELNSTPEILDEPEVKIPTDQEIIDAHKIYSSDLNFNPSTGNAYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 37 - protein, 261 aa, Clostridioides difficile
PTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 38 - protein, 261 aa, Clostridioides difficile; identical to SEQ ID NO: 37

SEQ ID NO: 39 - protein, 261 aa, Clostridioides difficile
PTYFTNFDDYNNYPSTWSNVNTTNQDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIMDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 40 - protein, 261 aa, Clostridioides difficile; identical to SEQ ID NO: 39

(19) United States
(12) Patent Application Publication
(10) Pub. No.: US 2026/0151471 A1
(43) Pub. Date: Jun. 4, 2026

(54) VACCINE CONTAINING RBD2 OF CDTB COMPONENT FROM BINARY TOXIN CDT OF CLOSTRIDIOIDES DIFFICILE

(71) Applicant: University of South Florida, Tampa, FL (US)

(72) Inventors: Xingmin Sun, Tampa, FL (US); Shaohui Wang, Tampa, FL (US)

(21) Appl. No.: 19/459,607

(22) Filed: Jan. 26, 2026

Related U.S. Application Data
(63) Continuation-in-part of application No. PCT/US2024/039462, filed on Jul. 25, 2024.
(60) Provisional application No. 63/516,416, filed on Jul. 28, 2023.

(51) Int. Cl. A61K 39/08; A61K 38/00; A61K 39/00; A61P 31/04; C07K 14/33; C07K 16/1282

(52) U.S. Cl. CPC A61K 39/08; A61P 31/04; C07K 14/33; C07K 16/1282; A61K 38/00; A61K 2039/55505; A61K 2039/575

(57) ABSTRACT

A novel vaccine and methods of preventing and treating C. difficile infection in a patient is described. The vaccine is comprised of at least a portion of receptor binding domain 2 (RBD2) protein or a protein comprising at least a portion of receptor binding domain 1 (RBD1) protein and RBD2 protein (RBD1+2) from binary toxin (CDT) of C. difficile. Administration of the vaccine, as well as anti-RBD2 or anti-RBD1+2 serum, has been shown to prevent C. difficile infection as well as treat existing infections.

### CROSS REFERENCE TO RELATED APPLICATIONS

This application is a continuation in part of and claims priority to International Application No. PCT/US2024/039462, entitled “Vaccine Containing RBD2 of CDTB Component from Binary Toxin CDT of Clostridioides difficile, filed Jul. 25, 2024, which is a nonprovisional of and claims priority to U.S. Provisional Patent Application Ser. No. 63/516,416, entitled “Receptor binding domain 2 of CDTb component of the Clostridioides difficile binary toxin CST as a vaccine component against C. difficile infection”, filed Jul. 28, 2023, the contents of which are hereby incorporated by reference into this disclosure.

### GOVERNMENT SUPPORT

This invention was made with Government support under Grant Nos. RO1-AI132711 awarded by the National Institute of Health. The Government has certain rights in the invention.

### SEQUENCE LISTING

The sequence listing entitled “Vaccine Containing RBD2 from CDTb Component of Binary Toxin CDT of Clostridioides difficile” in XML format, created on Jul. 19, 2024 and being 4900 bytes in size, is hereby incorporated by reference into this disclosure.

### FIELD OF INVENTION

This invention relates to treatment and/or prevention of C. difficile infection. Specifically, the invention provides a novel protein vaccine containing the RBD2 of CDTb of binary toxin CDT and associated method of preventing and/or treating a bacterial infection such as C. difficile.

### BACKGROUND OF THE INVENTION

Clostridioides difficile (C. difficile) is an anaerobic, spore-forming, Gram-positive anaerobic bacterium¹. Symptoms of C. difficile infection (CDI) range from diarrhea to intestinal inflammation and death, which are mainly caused by two protein toxins, toxin A (TcdA) and toxin B (TcdB)^{2,3}. In addition to TcdA and TcdB, between about 5-30% of C. difficile strains produce a third toxin termed binary toxin (CDT), which is encoded by the cdtA and cdtB genes^{4,5}. CDT is believed to enhance TcdA and TcdB toxicity and is related to severe disease and higher sporulation rates^{6,7}.

CDT is composed of the enzymatic component CDTa, and the binding and translocation component, CDTb, which mediates cell entry of CDTa⁸. CDTa, which is responsible for enzymatic activity, was found to be a two-domain enzyme: the N-terminal part (residues 1 to 215), which is important for CDTa binding to CDTb, and the C-terminal part (residues 224 to 420), which is important for enzyme's catalytic toxicity which catalyzes the ADP-ribosylation of actin^{9,10}.

CDTb is the binding and pore-forming component of the CDT. CDTb is synthesized as an inactive precursor form including SD (signaling domain), AD (activation domain), HD1 (heptamerization domain 1), βBD (β binding domain), HD2 (heptamerization domain 2), HD3 (heptamerization domain 3), RBD1 (receptor binding domain 1) and RBD2 (receptor binding domain 2)^{11,12} The pre-form CDTb undergoes autocleavage by removing SD and AD and matures into activated CDTb (mCDTb)¹³. Removal of SD and AD can be achieved by incubation of pre-form CDTb with trypsin in vitro.

Recent structural studies of CDTb showed that RBD1 domain lacks sequence homology to any other known toxin and was found to have a Ca²⁺-binding site, and RBD2 domain is not present in other members of this toxin family. Also, RBD2 was shown to be critical for establishing the di-heptamer macromolecular assembly in the activated CDTb that is necessary for host cell toxicity¹³. Deletion of RBD2 from CDTb, resulted in a heptameric structure with significantly reduced cytotoxicity. It was postulated that the RBD2 domain is crucial for the di-heptamer assembly as well as being critical for delivering toxic CDTa to host cells⁹. These studies map RBD2 bimolecular interactions for developing inhibitors targeting CDTb.

Recently, it is reported that CDTb in the absence of CDTa causes rounding of Vero cells and induces calcium-permeable trans-membrane pores in human CaCo-2 colon carcinoma cells¹⁴⁻¹⁶. Others have reported that CDTa is predicted to bind to a CDTb heptamer on the cell surface¹¹. Here, the inventors analyzed the cytotoxic effect of CDTa/b and evaluated the immunogenicity of RBD1 and RBD2 and their potential as effective vaccine components against CDI.

Given the lack of efficacy in current vaccines against C. difficile, what is needed is an efficacious vaccine against C. difficile infection (CDI).

### SUMMARY OF INVENTION

Symptoms of Clostridioides difficile infection (CDI) are mainly caused by two protein toxins, toxin A (TcdA), and toxin B (TcdB). In addition, between about 5-30% of C. difficile strains produce a third toxin termed binary toxin (CDT), which is believed to enhance TcdA and TcdB toxicity and is related to severe disease. CDT is composed of the enzymatic component CDTa, and the binding and translocation component CDTb, which mediates cell entry of CDTa. CDTb has two receptor binding domains (RBD1 and RBD2). Recent structural studies of CDTb showed that RBD2 domain was critical for establishing the di-heptamer macromolecular assembly in activated CDTb that is necessary for host cell toxicity. These studies assist in mapping RBD2 bimolecular interactions for developing inhibitors targeting CDTb. The inventors have analyzed the cytotoxic effect of CDTa/CDTb and evaluated the immunogenicity of RBD1 and RBD2. The data indicate that RBD2 can be an effective vaccine candidate to protect mice against CDT.

In an embodiment, a Clostridioides difficile (C. difficile) vaccine is presented comprising a therapeutically effective amount of a protein, or fragment thereof, from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2).

RBD2 may have a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

RBD1+2 may have a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

The vaccine may further comprise a pharmaceutically acceptable carrier and/or an adjuvant. In some embodiments, the adjuvant is an aluminum salt.

In a further embodiment, a method of preventing a C. difficile infection (CDI) in a patient in need thereof is presented comprising: administering to the patient a therapeutically effective amount of a therapeutic agent, the therapeutic agent comprising a serum containing antibodies to receptor binding domain 2 (RBD2) protein from binary toxin (CDT) of C. difficile; a serum containing antibodies to receptor binding domain 1 (RBD1) protein and RBD2 protein (RBD1+2) from the binary toxin (CDT) of C. difficile; or a vaccine, the vaccine comprising a therapeutically effective amount of a protein or fragment thereof from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2) and a pharmaceutically acceptable carrier, wherein the administration of the therapeutically effective amount of the therapeutic agent to the patient prevents CDI. The vaccine may further comprise an adjuvant, such as an aluminum salt.

In some embodiments, the therapeutic agent is the serum, more specifically the anti-RBD2 serum. In other embodiments, the therapeutic agent is the anti-RBD1+2 serum.

In other embodiments, the therapeutic agent is the vaccine. The vaccine may be comprised of the RBD2 having a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

Alternatively, the vaccine may be comprised of the RBD1+2 having a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

In a further embodiment, a method of treating a C. difficile infection (CDI) in a patient in need thereof comprising: administering to the patient a therapeutically effective amount of a therapeutic agent, the therapeutic agent comprising a serum containing antibodies to receptor binding domain 2 (RBD2) protein from binary toxin (CDT) of C. difficile; a serum containing antibodies to receptor binding domain 1 (RBD1) protein and RBD2 protein (RBD1+2) from the binary toxin (CDT) of C. difficile; or a vaccine, the vaccine comprising a therapeutically effective amount of a protein, or fragment thereof, from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2) and a pharmaceutically acceptable carrier, wherein the administration of the therapeutically effective amount of the therapeutic agent to the patient prevents CDI. The vaccine may further comprise an adjuvant, such as an aluminum salt.

In some embodiments, the therapeutic agent is the serum, more specifically the anti-RBD2 serum. In other embodiments, the therapeutic agent is the anti-RBD1+2 serum.

In other embodiments, the therapeutic agent is the vaccine. The vaccine may be comprised of the RBD2 having a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

Alternatively, the vaccine may be comprised of the RBD1+2 having a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

In another embodiment, a kit for preventing and/or treating CDI is presented comprising an immunogenic composition comprising a therapeutically effective amount of a protein, or fragment thereof, from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2) and a pharmaceutically acceptable carrier. The kit may optionally include an adjuvant such as an aluminum salt. Instructions for use may be included in the kit.

The RBD2 may have a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

The RBD1+2 having a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

In other embodiments, the kit for preventing and/or treating CDI is comprised of serum containing antibodies to receptor binding domain 2 (RBD2) protein from binary toxin (CDT) of C. difficile, i.e., anti-RBD2 serum, and/or a serum containing antibodies to receptor binding domain 1 (RBD1) protein and RBD2 protein (RBD1+2) from the binary toxin (CDT) of C. difficile, i.e., anti-RBD1+2 serum. A pharmaceutically acceptable carrier may also be present with the serum and instructions may be included in the kit. The kit may optionally include an adjuvant such as an aluminum salt.

In further embodiments, the kit may contain both a protein composition and a serum composition containing antibodies as described above.

In another embodiment, a method of inducing an immune response in a patient having a bacterial infection is presented comprising administering to the patient a therapeutically effective amount of a therapeutic agent selected from the group consisting of a serum containing antibodies to receptor binding domain 2 (RBD2) protein from binary toxin (CDT) of C. difficile, i.e., anti-RBD2 serum; a serum containing antibodies to receptor binding domain 1 (RBD1) protein and RBD2 protein (RBD1+2) from the binary toxin (CDT) of C. difficile, i.e., anti-RBD1+2 serum; and an immunogenic composition comprising a therapeutically effective amount of a protein, or fragment thereof, from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2) and a pharmaceutically acceptable carrier, wherein the administration of the therapeutic agent induces increases IgG and/or IgA levels as compared to a control.

In embodiments in which an immunogenic composition comprised of a protein is used, the RBD2 may have a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

Alternatively, in immunogenic compositions in which RBD1+2 are used, RBD1+2 having a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

### BRIEF DESCRIPTION OF THE DRAWINGS

For a fuller understanding of the invention, reference should be made to the following detailed description, taken in connection with the accompanying drawings, in which:

FIG. 1 is an image depicting CDTb phylogeny. Amino acid sequences were aligned with the Muscle algorithm in MegaX before computing a Maximum Likelihood tree with 500 bootstrap replicates (bootstrap values >50 displayed). The ribotype of each source strain is displayed adjacent to the strain name and is color-coded for easier identification.

FIG. 2 is an image depicting CDTb domain homology. Jalview software was used to visualize MUSCLE alignments of CDTb sequences. Conservation scores between 0 (no agreement) and 11 (identical amino acids) were reported for each amino acid position as determined through Jalview. The ribotype each source strain is displayed adjacent to the strain name and is color-coded for easier identification.

FIG. 3 is an image depicting RBD2 homology. Jalview software was used to visualize MUSCLE alignments of CDTb sequences. Conservation scores between 0 (no agreement) and 11 (identical amino acids) were reported for each amino acid position as determined through Jalview. The ribotype each source strain is displayed adjacent to the strain name and is color-coded for easier identification.

FIG. 4A-C are a series of images depicting (A) Domains of CDTa and CDTb. CDTb is synthesized as an inactive pro-form precursor including SD (signaling domain), AD (activation domain), HD1 (heptamerization domain 1), βBD (β binding domain), HD2 (heptamerization domain 2), HD3 (heptamerization domain 3), RBD1 (receptor binding domain 1) and RBD2 (receptor binding domain 2). The pre-form CDTb undergoes autocleavage by removing SD and AD and mature into activated CDTb (mCDTb). Removal of SD and AD can be achieved by incubation of pre-form CDTb with trypsin in vitro. (B) Expression and purification of CDTa and CDTb. Gene sequence encoding CDTa and CDTb were cloned into E. coli. Protein CDTa and CDTb were purified from bacterial lysate by Ni-affinity chromatography and analyzed by SDS-PAGE. CDTb was activated by incubation with 0.2 μg of trypsin/μg of protein for 30 minutes at 37° C., generating activated CDTb (mCDTb). (C) Expression and purification of RBD1, RBD2 and RBD1+2 of CDTb. Gene sequence encoding RBD1, RBD2 and RBD1+2 were cloned into E coli. Protein RBD1, RBD2 and RBD1+2 were purified from bacterial lysate by Ni-affinity chromatography and analyzed by SDS-PAGE.

FIG. 5A-B is a series of graphs depicting CDT-mediated cell rounding. (A) CT26 cells were treated with increasing concentrations of CDTa, CDTb, or mCDTb alone or in combination (CDTa/mCDTb: 1/7) at 37° C. for 2 h. For control, cells were left untreated (control). (B) Percentage of rounded cells was determined from images. Values are given as mean±SEM (n=3 images per condition). Experiments were repeated trice, and results from one representative experiment are shown. Significance was determined using student's t test (**p≤0.01, ****p≤0.0001, ns=not significant, vs. control).

FIG. 6A-B are a series of graphs depicting the predicted immunogenic regions (in yellow) of RBD1 and RBD2. B cell epitopes of (A) RBD1 and (B) RBD2 were predicted with the BepiPred-2.0 server. The residues with scores above the threshold (default value is 0.5) are predicted to be part of an epitope and colored in yellow on the graph (where Y-axes depicts residue scores and X-axes residue positions in the sequence). RBD2 contains 5 immunogenic peptides (yellow).

FIG. 7A-H is a series of images depicting (A and B) RBD1 or RBD1+2 immunizations induce anti-RBD1 antibody response as shown in sera. (C and D) RBD2 or RBD1+2 immunizations induce anti-RBD2 antibody responses as shown in sera. (E and F) RBD1 and RBD1+2 immunizations induce anti-RBD1 antibody responses as shown in feces. (G and H) RBD2 and RBD1+2 immunizations induce anti-RBD2 antibody responses as shown in feces. Groups of C57BL/6 mice (n=5) were immunized 3 times at 12-day intervals via i.p. route with 10 μg of RBD1, RBD2 or RBD1+2 in the presence of alum as an adjuvant. Sera and feces were collected, and anti-RBD1 and anti-RBD1 and anti-RBD2 IgG/IgA titers measured by standard ELISA. Values are given as mean±SEM. Significance was determined using student's t test (*p<0.05 vs 1^{st} IM).

FIG. 8A-D is a series of graphs depicting immunization with (A-D) RBD2 or RBD1+2, not RBD1 (IM route) induces significant anti-CDTb antibody responses in mice. Groups of C57 BL/6 mice (N=5) were immunized 3 times at 12-day intervals via IM route with 10 μg of RBD1, RBD2 or RBD1+2 in the presence of alum as an adjuvant. Sera were collected, and anti-CDTb IgA/G titers (A/B) measured by standard ELISA. (*p<0.05 vs 1^{st} immunization)

FIG. 9A-B are a series of graphs depicting (A) RBD2 or RBD1+2 immunization, but not RBD1 immunization protects mice against CDT challenge. Fourteen days after third immunization, mice from RBD1/RBD2/RBD1+2- or PBS-immunized group were i.p. challenged with lethal dosage of CDT (CDTa (260 ng/moue) plus mCDTb (2870 ng/mouse), n=5 for each group), monitored for survival and disease symptoms for 80 hrs. (B) Cleaved CDTb (mTcdB) only is not toxic in mice. Fourteen days after third immunization, mice from RBD1/RBD2/RBD1+2- or PBS-immunized group were i.p. challenged with mCDTb (2870 ng/mouse, n=5 for each protein group), monitored for survival and disease symptoms for 72 hrs.

FIG. 10A-B are a series of images depicting anti-RBD2 or anti-RBD1+2 serum inhibits CDT-mediated cell rounding. CT26 cells were treated with CDT (200 ng/ml of CDTa plus 2208 ng/ml of mCDTb, CDTa/CDTb is 1/7) in the presence of pre-immune serum or anti-RBD2/anti-RBD1/anti-RBD1+2 serum. Images (A) were taken at 2 h of treatment. (B) Percentage of rounded cells was determined from images. Values are given as mean±SEM (n=3 images per condition). Experiments were repeated trice, and results from one representative experiment are shown. Significance was determined using student's t test (****p≤0.0001, ns=not significant, vs. cells with no serum added).

FIG. 11A-B is a series of graphs depicting immunizations with RBD2 induce significant anti-RBD2 antibody responses. Groups of Golden Syrian hamsters (n=6) were immunized 3 times at 12-day intervals via intraperitoneal (i.p.) route with 10 μg of RBD2 with alum as an adjuvant. Sera were collected, and (A) anti-RBD2 IgG and (B) anti-RBD2 IgA titers in sera measured by standard ELISA. (*p<0.05 vs 1^{st} IM)

FIG. 12A-B are a series of graphs depicting immunizations with RBD2 provide hamsters significant protection against infection with C. difficile strain DSM101085. Two groups of hamsters (n=6) were immunized (i.p.) 3 times with RBD2 (10 ug/hamster/immunization) or PBS with alum as an adjuvant at 12-day intervals. Two weeks after the 3^{rd} immunization, hamsters were injected (i.p.) with 30 mg/kg clindamycin followed by challenge with C. difficile DSM101085 (1000 spores/hamster, via gavage), 1 day later. Kaplan-Meier survival plots (A) and mean relative weight of all surviving mice (up to the day of death) (B) of different groups are illustrated. Data were presented as mean relative weight±standard error (*p<0.05; **p<0.01).

FIG. 13A-F are a series of images depicting immunization with RBD2 provided significant protection to hamsters against infection with C. difficile DSM101085 strain. Groups of hamsters (N=10) were immunized with 30 ug RBD2 or PBS (200 μl) via IM route for 3 times at 14-day intervals. Sera and feces were collected after each immunization. Anti-RBD2 IgG/A titers in sera and feces were determined by ELISA (A, B, C and D). Seven days after the third immunization, hamsters were given clindamycin (IP, 30 mg/kg/day for 3 days), followed by infection with 2×10⁴ C. difficile DSM101085 spores by gavage. Hamsters were monitored; survival (E) weight loss (F) were recorded. Animal survivals were analyzed by Kaplan-Meier survival analysis with a log-rank test of significance; data were presented as mean relative weight±standard error. (*p<0.05, ***p<0.001)

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### ABBREVIATIONS

CDI—Clostridioides difficile infection
CDT—C. difficile binary toxin
CDTa—component A of binary toxin CDT
CDTb—component B of binary toxin CDT
LSR—lipolysis-stimulated lipoprotein receptor
RBD1—Receptor Binding Domain 1
RBD2—Receptor Binding Domain 2
RT—Ribotype

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used herein, the singular forms “a,” “an” and “the” include plural referents unless the context clearly dictates otherwise. For example, “a bacterium” includes “bacteria” or “plurality of bacteria.”

As used in this specification and the appended claims, the term “or” is generally employed in its sense including “and/or” unless the context clearly dictates otherwise.

All numerical designations, such as pH, temperature, time, concentration, and molecular weight, including ranges, are approximations which are varied up or down by increments of 1.0, 0.1, 0.01 or 0.001 as appropriate. It is to be understood, even if it is not always explicitly stated that all numerical designations are preceded by the term “about.” It is also to be understood, even if it is not always explicitly stated, that the reagents described herein are merely exemplary and that equivalents of such are known in the art and can be substituted for the reagents explicitly stated herein.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5 but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance that affect the novel characteristics of the invention as described herein. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means ±10% of the numerical.

As used herein “patient” is used to describe a mammal, preferably a human, to whom treatment is administered, including prophylactic treatment with the compositions of the present invention. Non-limiting examples of mammals include humans, rodents, aquatic mammals, domestic animals such as dogs and cats, farm animals such as sheep, pigs, cows, and horses. “Patient” and “subject” are used interchangeably herein.

“Administering” or “administration” as used herein refers to the process by which the compositions of the present invention are delivered to the patient. The compositions may be administered in various ways, including but not limited to, enteral and parenteral routes, namely, orally, rectally, nasally, subcutaneously, intradermally, parenterally such as intravenously, intraperitoneally, and intramuscularly, although other enteral and parenteral routes are contemplated including through the mucosa.

“Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articular, subcapsular, intrathecal, intraventricular, intracisternal, intranigral, subarachnoid, intraspinal, and intrasternal injection and infusion. Dosing can be by any suitable route, e.g., by injections, such as intravenous or subcutaneous injections, depending in part on whether the administration is brief or chronic. Various dosing schedules including but not limited to, single or multiple administrations over various time-points, bolus administration, and pulse infusion are contemplated herein.

A “therapeutic agent” as used herein refers to a substance, composition, compound, chemical, component, or extract that has measurable specified or selective physiological activity when administered to an individual in a therapeutically effective amount. In some embodiments, the therapeutic agent may be an antibacterial composition. Examples of therapeutic agents as used in the present invention include, but are not limited to, proteins and serum derived from such proteins. At least one therapeutic agent is used in the compositions of the present invention, however in some embodiments, multiple therapeutic agents are used. In some embodiments, the novel therapeutic agent described herein may be combined with another therapeutic agent that targets a different area of the bacteria or targets a different disease target. In some embodiments, one or more therapeutic agents may be encapsulated within a nanoparticle. In some embodiments, the therapeutic agent is used to treat an infection such as a bacterial infection like that from a Gram positive bacteria like C. difficile.

A “vaccine” as used herein refers to an antigenic or immunogenic composition usually comprising an infectious factor or a portion of an infectious factor, such as an antigen, in combination with an immune adjuvant, administered into the body to elicit an immune response such that resistance to new infection is enhanced and/or clinical severity of the disease is reduced. The antigenic portion may be a microorganism, such as a virus or bacterium, or a portion thereof, a natural product purified from a microorganism, or a portion thereof, or a synthetic or genetically engineered protein, peptide, polysaccharide, or similar product, or a portion thereof. In some embodiments, the antigenic portion of the vaccine of the present invention is comprised of a portion of the binding and translocation component (CDTb) of the binary toxin (CDT) of C. difficile. In some embodiments, the antigenic portion of the vaccine is comprised of at least a portion of receptor binding domain 2 (RBD2). In other embodiments, the antigenic portion of the vaccine is comprised of at least a portion of the combination of receptor binding domain 1 (RBD1) and RBD2, denoted RBD1+2.

The term “immunogenic composition” refers to a composition that comprises at least one antigen, which elicits an immunological response in the host to which the immunogenic composition is administered. Such immunological response may be a cellular and/or antibody-mediated immune response to the immunogenic composition of the invention. Preferably, the immunogenic composition induces an immune response and, more preferably, confers protective immunity against one or more of the clinical signs of C. difficile infection.

An “immune” or “immunological response” as used herein includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the subject will display either a protective immunological response or a therapeutically response.

A “protective immunological response” or “protective immunity” is demonstrated by either a reduction or lack of clinical signs normally displayed by an infected host, a quicker recovery time and/or a lowered duration of infectivity or lowered pathogen titer in the tissues or body fluids or excretions of the infected patient.

The terms “reduce or inhibit” as used herein refers to the ability to cause an overall decrease of at least 10%, 20%, 30%, 40%, 50%, 60%, 70%, 75%, 80%, 85%, 90%, 95%, or greater. Reduce or inhibit can refer, for example, to the symptoms of the disorder being treated or the presence, growth, adhesion, or spread of bacteria.

In some embodiments, receptor binding domain 1 (RBD1) comprises the amino acid sequence of any of SEQ ID NO:11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the amino acid sequence of any of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20 the foregoing percentages including each intervening value to the tenth of a percent. For example, at least 99.1%, 99.2%, 99.3%, etc. are contemplated for use herein. In some aspects of the invention, RBD1 is encoded by a nucleotide sequence, such as a cDNA, encoding an amino acid sequence having at least 80% homology with the amino acid sequence of any of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20 including all intervening amounts to the tenth, up to and including 100% homology.

In some embodiments, receptor binding domain 2 (RBD2) comprises the amino acid sequence of any of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the amino acid sequence of any of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, the foregoing percentages including each intervening value to the tenth of a percent. For example, at least 99.1%, 99.2%, 99.3%, etc. are contemplated for use herein. In some aspects of the invention, RBD2 is encoded by a nucleotide sequence, such as a cDNA sequence, encoding an amino acid sequence having at least 80% homology with the amino acid sequence of any of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30 including all intervening amounts to the tenth, up to and including 100% homology.

In some embodiments, a protein comprising both receptor binding domains, denoted as RBD1+2, comprises the amino acid sequence of any of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the amino acid sequence of any of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40, the foregoing percentages including each intervening value to the tenth of a percent. For example, at least 99.1%, 99.2%, 99.3%, etc. are contemplated for use herein. In some aspects of the invention, RBD1+2 is encoded by a nucleotide sequence, such as a cDNA sequence, encoding an amino acid sequence having at least 80% homology with the amino acid sequence of any of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40 including all intervening amounts to the tenth, up to and including 100% homology.

A “therapeutically effective amount” as used herein is defined as concentrations or amounts of components which are sufficient to effect beneficial or desired clinical results, including, but not limited to, any one or more of treating symptoms of bacterial infections, particularly C. difficile infection and preventing bacterial infection, particularly C. difficile infection. Compositions of the present invention can be used to effect a favorable change in the condition whether that change is an improvement, such as stopping, reversing, or reducing C. difficile infection, or a complete elimination of symptoms due to C. difficile infection. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. The dose may be adjusted according to response.

The dosing of compounds and compositions to obtain a therapeutic or prophylactic effect is determined by the circumstances of the patient, as is known in the art. The dosing of a patient herein may be accomplished through individual or unit doses of the compounds or compositions herein or by a combined or prepackaged or pre-formulated dose of a compounds or compositions.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a week or shorter up to about a year or longer. For example, the dosing regimen can be extended over a period of any of about 2, 3, 4, 5, 6, 7, 8, 9, 10, 11, and 12 months. In some embodiments, there is no break in the dosing schedule. In some embodiments, the interval between each administration is no more than about a week.

The compounds used in the present invention may be administered individually, or in combination with or concurrently with one or more other compounds used against bacteria, including Gram-positive bacteria such as C. difficile. Additionally, compounds used in the present invention may be administered in combination with or concurrently with other therapeutics for bacterial infections. In some embodiments, the vaccine compositions described herein may be administered with vaccines or other immunogenic compositions targeting Toxin A (TcdA) and/or Toxin B (TcdB) of C. difficile.

“Prevention” or “preventing” or “prophylactic treatment” as used herein refers to any of: halting the effects of bacterial infection, reducing the effects of bacterial infection, reducing the incidence of bacterial infection, reducing the development of bacterial infection, delaying the onset of symptoms of bacterial infection, increasing the time to onset of symptoms of bacterial infection, and reducing the risk of development of bacterial infection. In some embodiments, the bacterial infection is C. difficile.

“Treatment” or “treating” as used herein refers to any of the alleviation, amelioration, elimination, and/or stabilization of a symptom, as well as delay in progression of a symptom of a particular disorder. For example, “treatment” of bacterial infection may include any one or more of the following: amelioration and/or elimination of one or more symptoms associated with bacterial infection, reduction of one or more symptoms of bacterial infection, stabilization of symptoms of bacterial infection, and delay in progression of one or more symptoms of bacterial infection. In some embodiments, the bacterial infection is C. difficile.

“Infection” as used herein refers to the invasion of one or more microorganisms such as bacteria, viruses, fungi, yeast, or parasites in the body of a patient in which they are not normally present. In certain embodiments, the infection is from a bacteria such as a C. difficile.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include one or more diluents, adjuvants, and vehicles, as well as implant carriers, and inert, non-toxic solid or liquid fillers, or encapsulating material that does not react with the active ingredients of the invention. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, adjuvants, emulsifiers, salts for influencing osmotic pressure, buffers, coloring, and/or aromatic substances and the like which do not deleteriously react with the compositions, or polypeptides of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. “Diluents” may include water, saline, dextrose, ethanol, glycerol, and the like. Isotonic agents may include sodium chloride, dextrose, mannitol, sorbitol, and lactose, among others. Stabilizers include albumin and alkali salts of ethylendiamintetracetic acid, among others. In some embodiments, an adjuvant is used in addition to a pharmaceutically acceptable carrier in which the adjuvant may be an aluminum salt such as aluminum hydroxide, aluminum phosphate, alum (potassium aluminum sulfate), or mixed aluminum salts. Formulations are described in a number of sources that are well known and readily available to those skilled in the art. For example, Remington's Pharmaceutical Sciences (Martin E W [1995] Easton Pennsylvania, Mack Publishing Company, 19^{th} ed.) describes formulations which can be used in connection with the subject invention.

For ease of administration, the subject compounds may be formulated into various pharmaceutical forms. As appropriate compositions there may be cited all compositions usually employed for systemically or topically administering drugs. To prepare the pharmaceutical compositions of this invention, the protein, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably for administration nasally, orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. In particular, carriers for parenteral administration include aqueous solutions of dextrose, saline, pure water, ethanol, glycerol, propylene glycol, peanut oil, sesame oil, polyoxyethylene-block polymers, and the like. Injectable solutions, for example, may be prepared in which the carrier comprises saline solution, glucose solution or a mixture of saline and glucose solution.

The compositions of the invention can further include at least one adjuvant. Adjuvants contain agents that can enhance the immune response against substances that are poorly immunogenic on their own (see, for example, Immunology Methods Manual, vol. 2, I. Lefkovits, ed., Academic Press, San Diego, Calif, 1997, ch. 13). Immunology Methods Manual is available as a four volume set, (Product Code Z37,435-0); on CD-ROM, (Product Code Z37,436-9); or both, (Product Code Z37,437-7). Adjuvants can be, for example, mixtures of natural or synthetic compounds that, when administered with compositions of the invention, such as proteins that stimulate a protective immune response made by the methods described herein, further enhance the immune response to the protein. Compositions that further include adjuvants may further increase the protective immune response stimulated by compositions of the invention by, for example, stimulating a cellular and/or a humoral response (i.e., protection from disease versus antibody production). Adjuvants can act by enhancing protein uptake and localization, extend or prolong protein release, macrophage activation, and T and B cell stimulation. Adjuvants for use in the methods and compositions described herein can be mineral salts, oil emulsions, mycobacterial products, saponins, synthetic products and cytokines. Adjuvants can be physically attached (e.g., linked by recombinant technology, by peptide synthesis or chemical reaction) to a composition described herein or admixed with the compositions described herein. In some embodiments, the adjuvant is an aluminum salt such as aluminum hydroxide, aluminum phosphate, alum (potassium aluminum sulfate), or mixed aluminum salts.

The terms “nucleic acid” or “nucleic acid sequence” or “nucleotide sequence” or “polynucleotide” are used interchangeably herein and refer to polynucleotides including DNA molecules, RNA molecules, cDNA molecules, or derivatives. The term encompasses single as well as double stranded polynucleotides. The nucleic acid of the present invention encompasses isolated polynucleotides (i.e., isolated from its natural context) and genetically modified forms. Moreover, comprised are also chemically modified polynucleotides including naturally occurring modified polynucleotides such as glycosylated or methylated polynucleotides or artificial modified one such as biotinylated polynucleotides. Further, the terms “nucleic acid” and “polynucleotide” are interchangeable and refer to any nucleic acid. The terms “nucleic acid” and “polynucleotide” also specifically include nucleic acids composed of bases other than the five biologically occurring bases (adenine, guanine, thymine, cytosine, and uracil).

Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer, or tetramer. Polypeptides may also comprise single chain polypeptides or multichain polypeptides, such as antibodies or insulin, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments “variant mimics” are provided. A “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Orthologs” refers to genes in different species that evolved from a common ancestral gene by speciation. Normally, orthologs retain the same function in the course of evolution. Identification of orthologs is important for reliable prediction of gene function in newly sequenced genomes.

“Analogs” is meant to include polypeptide variants that differ by one or more amino acid alterations, for example, substitutions, additions, or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions, and covalent modifications with respect to reference sequences, in particular the polypeptide sequences disclosed herein, are included within the scope of this disclosure as indicated. For example, sequence tags or amino acids, such as one or more lysines, can be added to peptide sequences (e.g., at the N-terminal or C-terminal ends). Sequence tags can be used for peptide detection, purification, or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

As used herein, the term “homology” refers to the overall relatedness between polymeric molecules, e.g., between nucleic acid molecules (e.g., DNA molecules and/or RNA molecules) and/or between polypeptide molecules. Polymeric molecules (e.g., nucleic acid molecules (e.g., DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

As used herein, “percentage of sequence identity” or “percentage of homology” means the value determined by comparing two optimally aligned sequences, wherein the portion of the polynucleotide sequence may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′- or 3′-terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence.

The term “substantial identity” or “substantial homology” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

The term “vector” is well known to the person skilled in the art. The term “vector” as it is known in the art refers to a polynucleotide construct, typically a plasmid or a virus, used to transmit genetic material to a host cell. Vectors may be, for example, viruses, plasmids, cosmids, or phage. A vector as used herein may be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The expression vectors useful in the present invention are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional sequences, such as sequences encoding a protein.

The selected nucleotide sequence is operably linked to control elements that direct the transcription or expression thereof in the subject in vivo. Such control elements can comprise control sequences normally associated with the selected gene. Alternatively, heterologous control sequences can be employed. The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a Rous sarcoma virus (RSV) promoter, pol II promoters, pol III promoters, synthetic promoters, hybrid promoters, and the like. In addition, sequences derived from nonviral genes, such as the murine metallothionein gene, will also find use herein. Such promoter sequences are commercially available.

The term “promoter,” as used herein refers to a region or regions of a nucleic acid sequence that permits binding of RNA polymerase and directs transcription of a gene. Typically, a promoter is located in the 5′-non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter may be inducible, repressible, and/or constitutive. Inducible promoters initiate increased levels of transcription from DNA under their control in response to some change in culture conditions, such as a change in temperature.

As used herein, the term “enhancer” refers to a type of regulatory element that may increase the efficiency of transcription, regardless of the distance or orientation of the enhancer relative to the start site of transcription.

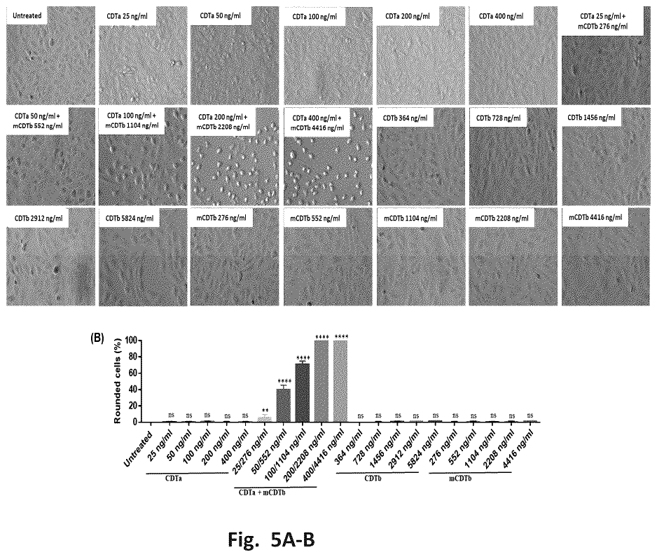

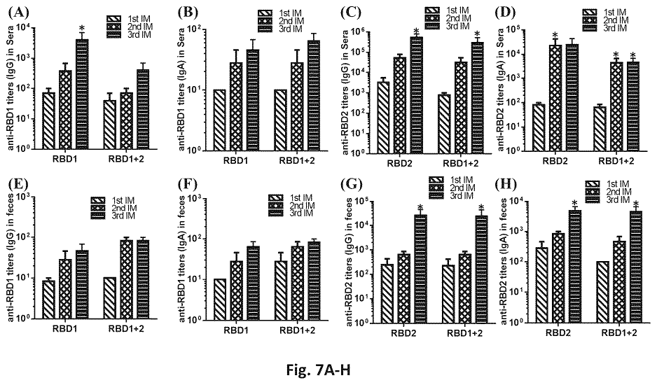

C. difficile binary toxin is an actin-ADP-ribosylating protein¹², and is encoded by the cdtA and cdtB genes, located within a 6.2 kb region designated the “CDT locus”^{12,18}. The lipolysis-stimulated lipoprotein receptor (LSR) was identified as the host cell receptor for CDT. CDTb first binds to this host cell receptor, then CDTa binds to CDTb and induces endocytosis into the host cell¹⁹. HeLa cells (no LSR expression) and CaCo-2 cells with LSR knock-out were not susceptible to be induced for cell rounding by CDTb alone, indicating that the binding of CDTb to LSR is essential for the observed CDTb-mediated effects on the cell rounding¹⁴. The inventors discovered that mouse intestinal epithelial CT26 cells were also not susceptible to be induced for cell rounding by CDTb or activated CDTb alone (FIG. 5), indicating that CT26 cells may not express LSR. No mice died when challenged with activated CDTb (mCDTb) without CDTa (FIG. 7), which is in agreement with a previous report²⁰.

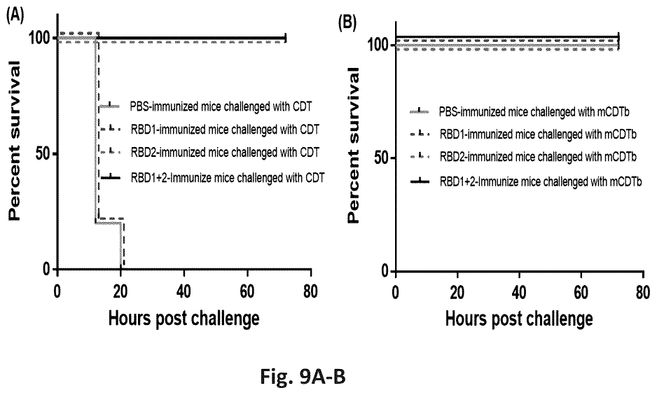

There are two receptor binding domains, RBD1 and RBD2, in the CDTb¹³. RBD2 was shown to be critical in activated CDTb that is necessary for the host cell toxicity, and RBD1 was found to have a Ca²⁺-binding site. To evaluate immunogenicity of RBD1 and RBD2, the inventors expressed RBD1, RBD2 and RBD1+2, and found that all three proteins induced significantly high levels of IgG antibody responses against RBD1 or RBD2 in sera (FIG. 7). However, only RBD2, not RBD1, induced immune protection against CDT (FIGS. 9 & 10), confirming the essential role of RBD2 in CDTb-mediated cell binding and CDT toxicity¹³, while validating RBD2 as a vaccine component against CDI.

### Results

Homology of CDTb and RBD2 Among Major Toxinotypes and Ribotypes of C. difficile Strains

An effective vaccine candidate should be conserved in different ribotypes (RTs) of C. difficile strains. To this end, the inventors investigated the homology of CDT and RBD2 among major C. difficile toxinotypes (TcdA⁺TcdB⁺CDT⁺, TcdA⁻TcdB⁺CDT⁺, TcdA⁻TcdB⁻CDT⁺) and RTs (RT027, RT078, RT066, RT126, RT045, RT033, RT023, RT019, RT036, and RT244) expressing CDT (Table 1).

TABLE 1
C. difficile strains selected for CDTb analysis

| | | | Accession | Refer- |
|---|---|---|---|---|
| Ribotype | Strain | Database | number/Barcode | ences |
| 019 | C00002493 | Enterobase | CLO_AA3440AA | — |
| | SMG-20-1235 | Enterobase | CLO_CA2097AA | — |
| | SMG-20-1765 | Enterobase | CLO_CA7590AA | — |
| 023 | DSM 102859 | GenBank | CP020378.1 | ³⁸ |
| | C00002723 | Enterobase | CLO_AA2553AA | — |
| | C00001506 | Enterobase | CLO_AA3450AA | — |
| 027 | R20291 | GenBank | FN545816.1 | ³⁹ |
| | CD-17-01474 | GenBank | NZ_CP026591.1 | ⁴⁰ |
| | DSM 27638 | GenBank | CP011846.1 | ⁴¹ |
| 033 | IS58 | Enterobase | CLO_AA5262AA | — |
| | RPH0101 | Enterobase | CLO_AA7921AA | — |
| | OCD52 | Enterobase | CLO_BA0437AA | — |
| 036 | C00012856 | Enterobase | CLO_AA4832AA | — |
| | CCUG 20309 | GenBank | AF271719.1 | ⁴² |
| 045 | CD-16-00514 | Enterobase | CLO_AA3441AA | — |
| | SIRN_HG-021 | Enterobase | CLO_CA2840AA | — |
| | C00002490 | Enterobase | CLO_AA3441AA | — |
| 066 | C08-686 | Enterobase | CLO_AA5240AA | — |
| | CD-16-00440 | Enterobase | CLO_BA9576AA | — |
| | SMG-21-2330 | Enterobase | CLO_CA8141AA | — |
| 078 | TW11-RT078 | GenBank | CP035499.1 | ⁴³ |
| | R2 | GenBank | CP026614.2 | ⁴⁴ |
| | DSM 29747 | GenBank | CP019864.1 | ³⁸ |
| 126 | Dsm 29020 | GenBank | CP012325.1 | ³⁸ |
| | CD-16-00082 | Enterobase | CLO_AA4352AA | — |
| | 6058625 | Enterobase | CLO_AA5219AA | — |
| 244 | C00009695 | Enterobase | CLO_AA0309AA | — |
| | C00009691 | Enterobase | CLO_AA0310AA | — |
| | C00009694 | Enterobase | CLO_AA0311AA | — |

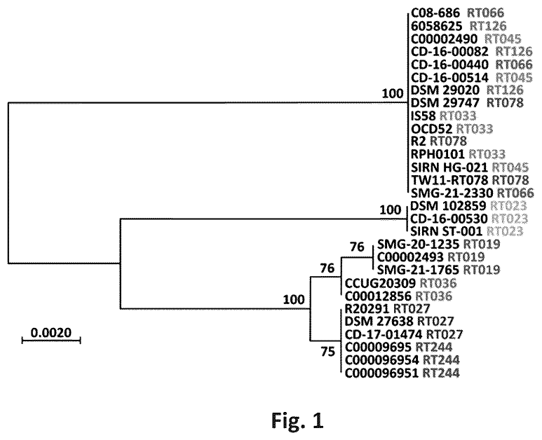

A maximum likelihood phylogenetic tree was generated using CDTb amino acid sequences from representative strains of CDT+ ribotypes (FIG. 1). The results suggest that there is, to some extent, a correlation between ribotype and CDTb similarity. Firstly, RT023 CDTb sequences are identical, and they form a unique cluster in the phylogenetic tree separate from the sequences sourced from other ribotypes. The same result is observed for RT019 and RT0360 CDTb, respectively. In some cases, multiple ribotypes share identical CDTb sequences. For example, the CDTb sequences of RT033, RT045, RT066, RT078, and RT126 representative strains were virtually identical. RT027 CDTb and RT244 CDTb sequences also cluster together in a similar manner. Nonetheless, all CDTb sequences from a given ribotype are clustered with the other sequences of that ribotype, suggesting that CDTb sequences are conserved within a given ribotype at the amino acid level. Since only two to three representative sequences were chosen for each ribotype, a larger-scale analysis could reveal exceptions to this trend.

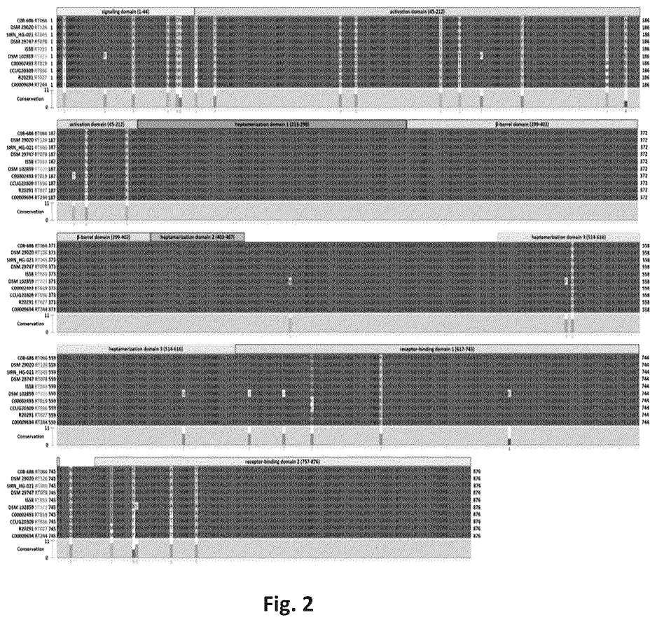

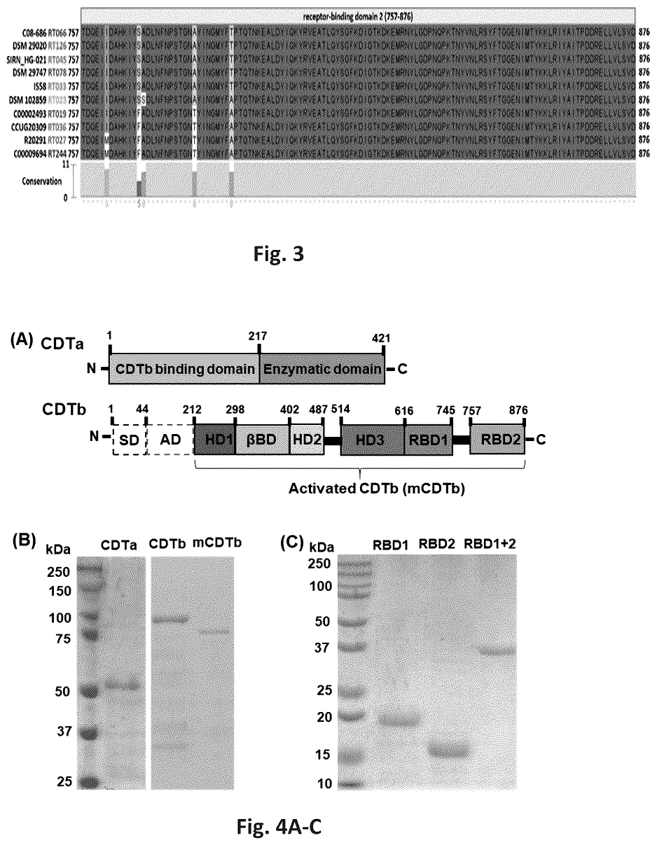

To more thoroughly examine CDTb sequence diversity, the inventors aligned the sequences of the whole CDTb protein (FIG. 2) and RBD2 (FIG. 3) using MUSCLE and visualized the output in Jalview software. Domains were annotated based on a prior review¹⁷. Sequence variations were most concentrated in the signaling domain (1 variation/7.33 residues) followed by the activation domain (1 variation/12.92 residues), RBD2 (1 variation/20 residues), RBD1 (1 variation/25.8 residues), and the heptamerization domain 3 (1 variation/34.33 residues). The heptamerization domain 1, heptamerization domain 2, and β-barrel domain were completely conserved between the examined strains. Regarding the RBD2, in particular, the 6 sequence variations are concentrated towards the N-terminal end of the RBD2 domain (residues 762-789), whereas the C-terminal end is highly conserved. In summary, both CDTb1 and RBD2 are highly conserved in C. difficile strains.

### CDT Causes Cell Rounding in a Concentration-Dependent Manner.

CDTa or CDTb or activated CDTb (mCDTb) alone does not induce CT26 cell rounding at all concentrations tested. The combination of CDTa plus mCDTb applied in concentrations of 100 ng/ml CDTa plus 1104 ng/ml mCDTb (CDTa:mCDTb at 1:7 molecular ratio) or higher led to the well-characterized rounding of CT26 cells after 2 h of incubation (FIG. 5A). The percentage of rounded cells was determined from cell images (FIG. 5B).

### Both RDB1 and RBD2 are Highly Immunogenic Based on B Cell Epitope Analysis

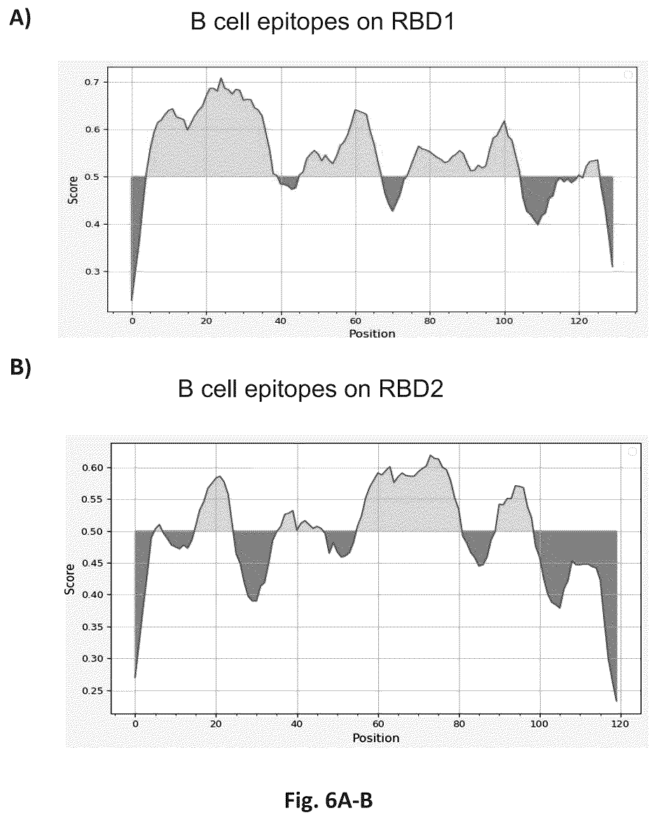

To predict the immunogenicity of RBD1 and RBD2, the inventors conducted B cell epitope analyses using the BepiPred 2.0 server. The prediction showed that both RBD1 and RBD2 are dominated by the immunogenic regions. (FIGS. 6A and B).

Immunization with RBD2 or RBD1+2 Induces Significant Anti-RBD2 or Anti-CDTb Antibody Responses and Protects Mice Against CDT Challenge

To evaluate the immunogenicity of RND1 and RBD2 and its potential against CDT, the inventors generated CDTa and CDTb. CDTa and CDTb (FIG. 4A) with a 6×His-tag (50 and 95 kDa) was expressed in E. coli BL21(DE3) and purified by Ni-affinity chromatography to a purity greater than 95% (FIG. 4B). Activation of CDTb was achieved by incubation with 0.2 μg of trypsin/μg of protein for 30 minutes at 37° C. (FIG. 4B). To evaluate the immunogenicity of RBD1 and RBD2 in vivo, the inventors generated RBD2 and RBD1 as well as their fusion RBD1+2 as comparators. RBD1, RBD2 or RBD1+2 with a 6×His-tag (19, 17 and 33 kDa) was expressed in E coli BL21(DE3), and purified by Ni-affinity chromatography to a purity greater than 95% (FIG. 4C).

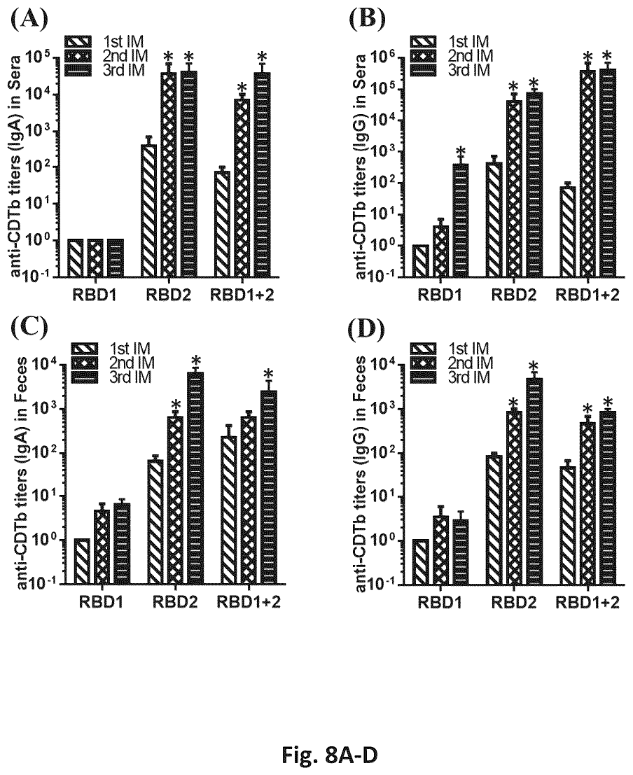

Immunizations of mice with 10 μg of RBD2 or RBD1+2 in combination with alum as an adjuvant via intramuscular route induced significant IgG/IgA antibody responses against RBD2 (FIG. 7) or CDTb (FIG. 8) in sera and feces. While immunization with RBD1 using the same antigen amount and adjuvant induced significant anti-RBD1 and anti-CDTb IgG antibody responses in sera (FIG. 7A, FIG. 8B), it did not induce significant anti-RBD1 IgG or IgA antibody responses in feces (FIGS. 7E &F), nor significant anti-CDTb IgG/IgA antibody responses in feces (FIG. 8) or anti-CDTb IgA antibody response in sera. These results suggest that RBD2 is more antigenic than RBD1.

Protection efficacy of RBD1, RBD2 or RBD1+2 immunization was evaluated in mice. After three immunizations (10 μg per immunization for 3 times at 12-day intervals), mice were challenged with a lethal dosage CDT (260 ng of CDTa/2870 ng of mCDTb). In vehicle (PBS)-immunized and RBD1-immunized groups, all mice died by 20 hours (FIG. 9A). In contrast, all RBD2-immunized mice, or RBD1+2-immunized mice survived (FIG. 9A). No mice died in all PBS-immunized mice or RBD1-, RBD2- or RBD1+2-immunized mice challenged with mCDTb alone (FIG. 9B), indicating that mCDTb is avirulent to mice and RND2 is more immunogenic and protective then RBD1 as an immunogen.

### Anti-RBD2 and Anti-RBD1+2 Serum Inhibits CDT-Mediated Cell Rounding.

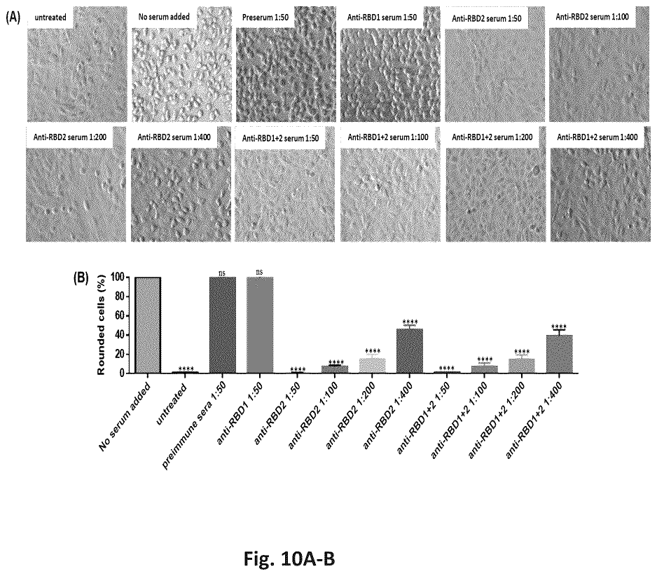

The inventors conducted experiments to determine if anti-RBD2 and anti-RBD1+2 serum could inhibit CDT-mediated cell rounding. Confluent CT26 cells were treated with CDT (200 ng/ml of CDTa plus 2208 ng/ml of mCDTb, CDTa/mCDTb=1/7) in the presence of pre-immune serum or anti-RBD2/anti-RBD1/anti-RBD1+2 serum for 2 h. As shown in FIG. 10, when the anti-RBD2 or anti-RBD1+2 serum was diluted 1 to 400 in the cells medium, cell rounding rate significantly decreased (45±5% or 39.33±6.67%, respectively, vs 100% for cells with no serum added). When the anti-RBD2/anti-RBD1+2 serum was diluted 1 to 50, CDT-mediated cell rounding was almost completely inhibited while anti-RBD1 serum was not.

Immunization with the Receptor Binding Domain 2 (RBD2) of CDTb Provides Hamsters Significant Protection Against Challenge with CDT-Producing-Only C. difficile Strain DSM101085

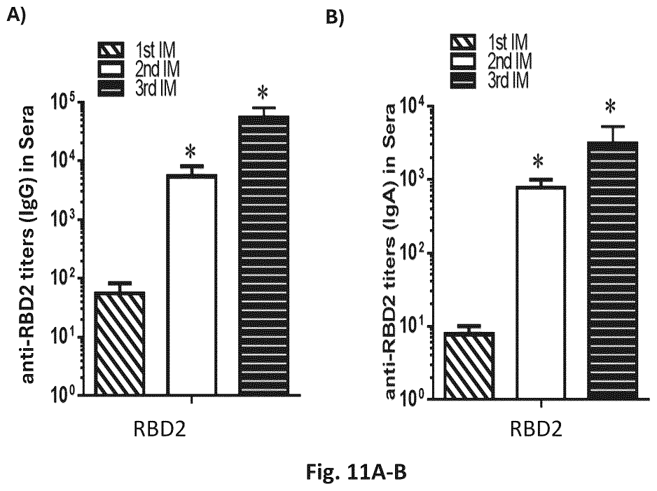

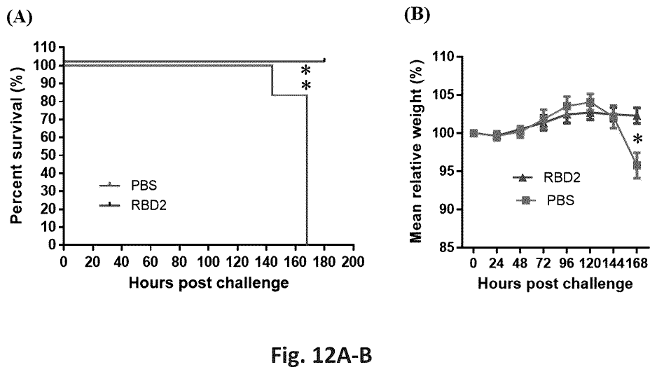

As noted above, the inventors have shown that immunizations with RBD2 of CDTb provide mice full protection against a lethal challenge with the binary toxin (CDT). It is known that mice are not susceptible to challenge with CDT-producing-only C. difficile strains. Since hamsters are highly susceptible to CDI, the inventors conducted experiments to determine whether immunizations with RBD2 could provide hamsters against challenge with a C. difficile strain producing CDT only. To this end, Golden Syrian hamsters (n=6) were immunized 3 times at 12-day intervals via i.p. route with 10 g of RBD2 with alum as an adjuvant. The data showed immunizations with RBD2 induced significant anti-RBD2 antibody responses (FIG. 11) and provided hamsters significant protection against infection with C. difficile strain DSM101085, which produces CDT only (FIG. 12).

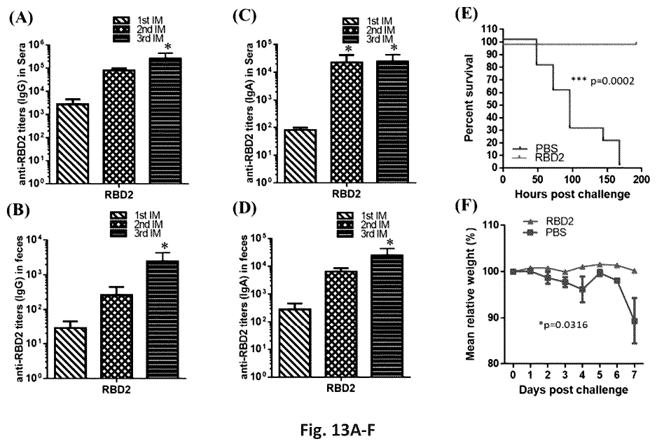

To further assess dosage, immunization of hamsters with RBD2 (30 ug per immunization per mice via intramuscular route) for 3 times at 14-day intervals induced significant levels of anti-RBD2 IgG or IgA antibodies in sera and feces (FIGS. 13A-D) and provided significant protection to hamsters against challenge with C. difficile DSM101085 strain (phenotype A-B-CDT+) spores infection at 2×10⁴ spores/hamster (FIGS. 13E-F).

### Materials and Methods

### Homology Analysis of CDTb and RBD2

CDT+ C. difficile ribotypes were chosen for analysis based on previous studies^{21,22}. A search was performed in Google Scholar and various genome databases including GenBank (National Center for Biotechnology Information) and the Enterobase Clostridioides database²³ to identify sequenced C. difficile strains from each ribotype (Table 1). Enterobase strain ribotypes were specified in each database entry, whereas the ribotypes of other strains were identified using literature sources²⁴⁻³⁰. At least two genomes were selected for analysis, with three genomes being used in most cases. CDTb amino acid sequences were mined from each genome before performing a MUSCLE alignment in MegaX software³¹ on default parameters. Then, a Maximum likelihood phylogenetic trees were constructed in MegaX with 500 bootstrap replicates. The cluster patterns of the phylogenetic trees were used to order the sequences of CdtB or the RBD2 domain of CdtB for a second MUSCLE alignment on the MPI Bioinformatics Toolkit server^{32,33}, as this application produced an output file suitable for visualization using Jalview³⁴. Jalview calculates conservation scores for MUSCLE alignments according to a previously defined algorithm³⁵ that assesses both the amino acid identity as well as physico-chemical properties of the amino acids at a given position to produce a score between zero (no similarities) and eleven (identical amino acids).

### Animals

All studies followed the Guide for the Care and Use of Laboratory Animals of the National Institutes of Health and were approved by the Institutes Animal Care and Use Committee (IACUC) at the University of South Florida. Wild-type C57BL/6 mice were purchased from Charles River Laboratories.

### Protein Expression and Purification

Gene sequences encoding the components of CDT (CDTa and CDTb), receptor binding domain 1 (RBD1), receptor binding domain 2 (RBD2) or RBD1 and RBD2 in combination (RBD1+2) from C. difficile R20291 were cloned into pET28a, expressed in E coli BL21 (DE3) with a N-terminal His-tag, and purified from bacterial lysate by Ni-affinity chromatography. CDTb was activated by incubation with 0.2 μg of trypsin/μg of protein for 30 minutes at 37° C. For immunization, RBD1, RBD2, and RBD1+2 proteins were further purified using an Endotoxin Removal Spin Column (Pierce™) to remove endotoxin following manufacturer's instruction.

Sequences for cdtB
cdtB of C. difficile C08-686 (RT066)
(residues 1-876)
(SEQ ID NO: 1)
MKVQMRNKKVLSFLTLTAIVSQALAYPVYAQTSTSSHSDNKKEIINEDIL
INNGLMGYYFTDEHFKDLKLMAPIKDGNLKFEEKKVDKLLNKDKSNVKSI
RWTGRIIPSKDGEYTLSTDRDDILMQVNNESTISNTLKVNMKKGKEYKFR
IELQDKNLGSIDNLSSPNLYWELDGIKKIIPAENLFLRDYSNIEKNDPFI
PNNNFFDPRLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQNPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSKLKPYKRYVFSGYSKDPLTSNSIIVKIKAKEEKTDYLVP
EQGYTKFSYEFETTEKDSSNIEITLIGSGTTYLDNLSITELNSTPEILNE
PEVKIPTDQEIIDAHKIYSADLNFNPSTGNAYINGMYFTPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile DSM 29020 (RT126)
(residues 1-876)
(SEQ ID NO: 2)
MKVQMRNKKVLSFLTLTAIVSQALAYPVYAQTSTSSHSDNKKEIINEDIL
INNGLMGYYFTDEHFKDLKLMAPIKDGNLKFEEKKVDKLLNKDKSNVKSI
RWTGRIIPSKDGEYTLSTDRDDILMQVNNESTISNTLKVNMKKGKEYKFR
IELQDKNLGSIDNLSSPNLYWELDGIKKIIPAENLFLRDYSNIEKNDPFI
PNNNFFDPRLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQNPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSKLKPYKRYVFSGYSKDPLTSNSIIVKIKAKEEKTDYLVP
EQGYTKFSYEFETTEKDSSNIEITLIGSGTTYLDNLSITELNSTPEILNE
PEVKIPTDQEIIDAHKIYSADLNFNPSTGNAYINGMYFTPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile SIRN-HG-021 (RT045)
(residues 1-876)
(SEQ ID NO: 3)
MKVQMRNKKVLSFLTLTAIVSQALAYPVYAQTSTSSHSDNKKEIINEDIL
INNGLMGYYFTDEHFKDLKLMAPIKDGNLKFEEKKVDKLLNKDKSNVKSI
RWTGRIIPSKDGEYTLSTDRDDILMQVNNESTISNTLKVNMKKGKEYKFR
IELQDKNLGSIDNLSSPNLYWELDGIKKIIPAENLFLRDYSNIEKNDPFI
PNNNFFDPRLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQNPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSKLKPYKRYVFSGYSKDPLTSNSIIVKIKAKEEKTDYLVP
EQGYTKFSYEFETTEKDSSNIEITLIGSGTTYLDNLSITELNSTPEILNE
PEVKIPTDQEIIDAHKIYSADLNFNPSTGNAYINGMYFTPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile DSM 29747 (RT078)
(residues 1-876)
(SEQ ID NO: 4)
MKVQMRNKKVLSFLTLTAIVSQALAYPVYAQTSTSSHSDNKKEIINEDIL
INNGLMGYYFTDEHFKDLKLMAPIKDGNLKFEEKKVDKLLNKDKSNVKSI
RWTGRIIPSKDGEYTLSTDRDDILMQVNNESTISNTLKVNMKKGKEYKFR
IELQDKNLGSIDNLSSPNLYWELDGIKKIIPAENLFLRDYSNIEKNDPFI
PNNNFFDPRLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQNPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSKLKPYKRYVFSGYSKDPLTSNSIIVKIKAKEEKTDYLVP
EQGYTKFSYEFETTEKDSSNIEITLIGSGTTYLDNLSITELNSTPEILNE
PEVKIPTDQEIIDAHKIYSADLNFNPSTGNAYINGMYFTPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile IS58 (RT033)
(residues 1-876)
(SEQ ID NO: 5)
MKVQMRNKKVLSFLTLTAIVSQALAYPVYAQTSTSSHSDNKKEIINEDIL
INNGLMGYYFTDEHFKDLKLMAPIKDGNLKFEEKKVDKLLNKDKSNVKSI
RWTGRIIPSKDGEYTLSTDRDDILMQVNNESTISNTLKVNMKKGKEYKFR
IELQDKNLGSIDNLSSPNLYWELDGIKKIIPAENLFLRDYSNIEKNDPFI
PNNNFFDPRLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQNPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSKLKPYKRYVFSGYSKDPLTSNSIIVKIKAKEEKTDYLVP
EQGYTKFSYEFETTEKDSSNIEITLIGSGTTYLDNLSITELNSTPEILNE
PEVKIPTDQEIIDAHKIYSADLNFNPSTGNAYINGMYFTPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile DSM 102859 (RT023)
(residues 1-876)
(SEQ ID NO: 6)
MKVQMRNKKVLSFLTFTAIVSQALVYPVYAQTSTSNHSNKKKEIINEDIL
PNNGLMGYYFTDEHFKDLKLMAPIKDGNLKFEEKKVDKLLDKDKSDVKSI
RWTGRIIPSKDGEYTLSTDRDDVLMQVNTESTISNKLKVNMKKGKEYKVR
IELQDKNLGSIDNLSSPNLYWELDGIKKIIPEENLFLRDYSNIEKNDPFI
PNNNFFDPKLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPMALN
TMDQFSSRLIPINYDQLKKLDAGKQIKLETTQVSGNFGTKNSSGQIVTEG
NSWSDYISQIDSISASIILDTENESYERRVTAKNFQDPEDKTPELTIGEA
IEKAFGATKKDGLLYFNDIPIDESCVELIFDDNTANKIKDSLKTLSDKQI
YNVKLERGMNILIKTPTYFINFDDYNNYPSIWSNVNTTNQDGLQGSANKL
NGETKIKIPMSELKPYKRYVFSGYSKDPLTSNSIIVKIKAKEEKTDYLVP
EQEYTKFSYEFETTEKDSSNIEITLIGSGTTYLDNLSITELNSTPEILDE
PEVKIPTDQEIIDAHKIYSSDLNFNPSTGNAYINGMYFAPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile C00002493 (RT019)
(residues 1-876)
(SEQ ID NO: 7)
MKIQMRNKKVLSFLTLTAIVSQALVYPVYAQTSTSNHSNKKKEIVNEDIL
PNNGLMGYYFTDEHFKDLKLMAPIKDGNLKFEEKKVDKLLDKDKSDVKSI
RWTGRIIPSKDGEYTLSTDRDDVLMQVNTESTISNTLKVNMKKGKEYKVR
IELQDKNLGSIDNLSSPNLYWELDGMKKIIPEENLFLRDYSTIEKDDPFI
PNNNFFDPKLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQDPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNKDGLQGSANKL
NGETKIKIPMSELKPYKRYVFSGYSKDPLTSNSIIVKIKAKEEKTDYLVP
EQGYTKFSYEFETTEKDSSNIEITLIGSGTTYLDNLSITELNSTPEILDE
PEVKIPTDQEIIDAHKIYFADLNFNPSTGNTYINGMYFAPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile CCUG20309 (RT036)
(residues 1-876)
(SEQ ID NO: 8)
MKIQMRNKKVLSFLTLTAIVSQALVYPVYAQTSTSNHSNKKKEIVNEDIL
PNNGLMGYYFTDEHFKDLKLMAPIKDGNLKFEEKKVDKLLDKDKSDVKSI
RWTGRIIPSKDGEYTLSTDRDDVLMQVNTESTISNTLKVNMKKGKEYKVR
IELQDKNLGSIDNLSSPNLYWELDGMKKIIPEENLFLRDYSNIEKDDPFI
PNNNFFDPKLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQDPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNKDGLQGSANKL
NGETKIKIPMSELKPYKRYVFSGYSKDPLTSNSIIVKIKAKEEKTDYLVP
EQGYTKFSYEFETTEKDSSNIEITLIGSGTTYLDNLSITELNSTPEILDE
PEVKIPTDQEIIDAHKIYFADLNFNPSTGNTYINGMYFAPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile R20291 (RT027)
(residues 1-876)
(SEQ ID NO: 9)
MKIQMRNKKVLSFLTLTAIVSQALVYPVYAQTSTSNHSNKKKEIVNEDIL
PNNGLMGYYFTDEHFKDLKLMAPIKDGNLKFEEKKVDKLLDKDKSDVKSI
RWTGRIIPSKDGEYTLSTDRDDVLMQVNTESTISNTLKVNMKKGKEYKVR
IELQDKNLGSIDNLSSPNLYWELDGMKKIIPEENLFLRDYSNIEKDDPFI
PNNNFFDPKLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQDPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSELKPYKRYVFSGYSKDPLTSNSIIVKIKAKEEKTDYLVP
EQGYTKFSYEFETTEKDSSNIEITLIGSGTTYLDNLSITELNSTPEILDE
PEVKIPTDQEIMDAHKIYFADLNFNPSTGNTYINGMYFAPTQTNKEALDY
IQKYRVEATLQYSGFKDIGTKDKEMRNYLGDPNQPKTNYVNLRSYFTGGE
NIMTYKKLRIYAITPDDRELLVLSVD
cdtB of C. difficile C00009694 (RT244)
(residues 1-876)
(SEQ ID NO: 10)
MKIQMRNKKVLSFLTLTAIVSQALVYPVYAQTSTSNHSNKKKEIVNEDIL
PNNGLMGYYFTDEHFKDLKLMAPIKDGNLKFEEKKVDKLLDKDKSDVKSI
RWTGRIIPSKDGEYTLSTDRDDVLMQVNTESTISNTLKVNMKKGKEYKVR
IELQDKNLGSIDNLSSPNLYWELDGMKKIIPEENLFLRDYSNIEKDDPFI
PNNNFFDPKLMSDWEDEDLDTDNDNIPDSYERNGYTIKDLIAVKWEDSFA
EQGYKKYVSNYLESNTAGDPYTDYEKASGSFDKAIKTEARDPLVAAYPIV
GVGMEKLIISTNEHASTDQGKTVSRATTNSKTESNTAGVSVNVGYQNGFT
ANVTTNYSHTTDNSTAVQDSNGESWNTGLSINKGESAYINANVRYYNTGT
APMYKVTPTTNLVLDGDTLSTIKAQENQIGNNLSPGDTYPKKGLSPLALN
TMDQFSSRLIPINYDQLKKLDAGKQIKLETTQVSGNFGTKNSSGQIVTEG
NSWSDYISQIDSISASIILDTENESYERRVTAKNLQDPEDKTPELTIGEA
IEKAFGATKKDGLLYFNDIPIDESCVELIFDDNTANKIKDSLKTLSDKKI
YNVKLERGMNILIKTPTYFTNFDDYNNYPSTWSNVNTTNQDGLQGSANKL
NGETKIKIPMSELKPYKRYVFSGYSKDPLTSNSIIVKIKAKEEKTDYLVP
EQGYTKFSYEFETTEKDSSNIEITLIGSGTTYLDNLSITELNSTPEILDE
PEVKIPTDQEIMDAHKIYFADLNFNPSTGNTYINGMYFAPTQTNKEALDY
IQKYRVEATLQYSGFKDIGTKDKEMRNYLGDPNQPKTNYVNLRSYFTGGE
NIMTYKKLRIYAITPDDRELLVLSVD

### Potential Sequences for Immunization

Exemplary sequences for RBD1, RBD2, and RBD1+2 are as follows:

RBD1 of C. difficile C08-686 (RT066)
(residues 617-745)
(SEQ ID NO: 11)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile DSM 29020 (RT126)
(residues 617-745)
(SEQ ID NO: 12)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile SIRN-HG-021 (RT045)
(residues 617-745)
(SEQ ID NO: 13)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile DSM 29747 (RT078)
(residues 617-745)
(SEQ ID NO: 14)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile IS58 (RT033)
(residues 617-745)
(SEQ ID NO: 15)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile DSM 102859 (RT023)
(residues 617-745)
(SEQ ID NO: 16)
PTYFINFDDYNNYPSIWSNVNTTNQDGLQGSANKLNGETKIKIPMSELKP
YKRYVFSGYSKDPLTSNSIIVKIKAKEEKTDYLVPEQEYTKFSYEFETTE
KDSSNIEITLIGSGTTYLDNLSITELNSTP
RBD1 of C. difficile C00002493 (RT019)
(residues 617-745)
(SEQ ID NO: 17)
PTYFTNFDDYNNYPSTWSNVNTTNKDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile CCUG20309 (RT036)
(residues 617-745)
(SEQ ID NO: 18)
PTYFTNFDDYNNYPSTWSNVNTTNKDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile R20291 (RT027)
(residues 617-745)
(SEQ ID NO: 19)
PTYFTNFDDYNNYPSTWSNVNTTNQDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTP
RBD1 of C. difficile C00009694 (RT244)
(residues 617-745)
(SEQ ID NO: 20)
PTYFTNFDDYNNYPSTWSNVNTTNQDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTP
RBD2 of C. difficile C08-686 (RT066)
(residues 757-876)
(SEQ ID NO: 21)
TDQEIIDAHKIYSADLNFNPSTGNAYINGMYFTPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile DSM 29020 (RT126)
(residues 757-876)
(SEQ ID NO: 22)
TDQEIIDAHKIYSADLNFNPSTGNAYINGMYFTPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile SIRN-HG-021 (RT045)
(residues 757-876)
(SEQ ID NO: 23)
TDQEIIDAHKIYSADLNFNPSTGNAYINGMYFTPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile DSM 29747 (RT078)
(residues 757-876)
(SEQ ID NO: 24)
TDQEIIDAHKIYSADLNFNPSTGNAYINGMYFTPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile IS58 (RT033)
(residues 757-876)
(SEQ ID NO: 25)
TDQEIIDAHKIYSADLNFNPSTGNAYINGMYFTPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile DSM 102859 (RT023)
(residues 757-876)
(SEQ ID NO: 26)
TDQEIIDAHKIYSSDLNFNPSTGNAYINGMYFAPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile C00002493 (RT019)
(residues 757-876)
(SEQ ID NO: 27)
TDQEIIDAHKIYFADLNFNPSTGNTYINGMYFAPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile CCUG20309 (RT036)
(residues 757-876)
(SEQ ID NO: 28)
TDQEIIDAHKIYFADLNFNPSTGNTYINGMYFAPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile R20291 (RT027)
(residues 757-876)
(SEQ ID NO: 29)
TDQEIMDAHKIYFADLNFNPSTGNTYINGMYFAPTQTNKEALDYIQKYRV
EATLQYSGFKDIGTKDKEMRNYLGDPNQPKTNYVNLRSYFTGGENIMTYK
KLRIYAITPDDRELLVLSVD
RBD2 of C. difficile C00009694 (RT244)
(residues 757-876)
(SEQ ID NO: 30)
TDQEIMDAHKIYFADLNFNPSTGNTYINGMYFAPTQTNKEALDYIQKYRV
EATLQYSGFKDIGTKDKEMRNYLGDPNQPKTNYVNLRSYFTGGENIMTYK
KLRIYAITPDDRELLVLSVD
RBD1+2 C. difficile C08-686 (RT066)
(residues 617-876)
(SEQ ID NO: 31)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTPEILNEPEVKIPTDQEIIDAH
KIYSADLNFNPSTGNAYINGMYFTPTQTNKEALDYIQKYRVEATLQYSGF
KDIGTKDKEMRNYLGDPNQPKTNYVNLRSYFTGGENIMTYKKLRIYAITP
DDRELLVLSVD
RBD1+2 C. difficile DSM 29020 (RT126)
(residues 617-876)
(SEQ ID NO: 32)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTPEILNEPEVKIPTDQEIIDAH
KIYSADLNFNPSTGNAYINGMYFTPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile SIRN-HG-021 (RT045)
(residues 617-876)
(SEQ ID NO: 33)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTPEILNEPEVKIPTDQEIIDAH
KIYSADLNFNPSTGNAYINGMYFTPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile DSM 29747 (RT078)
(residues 617-876)
(SEQ ID NO: 34)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTPEILNEPEVKIPTDQEIIDAH
KIYSADLNFNPSTGNAYINGMYFTPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile IS58 (RT033)
(residues 617-876)
(SEQ ID NO: 35)
PTYFTNFDDYNNYPSTWSNVNTTNQDGLQGSANKLNGETKIKIPMSKLKP
YKRYVFSGYSKDPLTSNSIIVKIKAKEEKTDYLVPEQGYTKFSYEFETTE
KDSSNIEITLIGSGTTYLDNLSITELNSTPEILNEPEVKIPTDQEIIDAH
KIYSADLNFNPSTGNAYINGMYFTPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile 102859 (RT023)
(residues 617-876)
(SEQ ID NO: 36)
PTYFINFDDYNNYPSIWSNVNTTNQDGLQGSANKLNGETKIKIPMSELKP
YKRYVFSGYSKDPLTSNSIIVKIKAKEEKTDYLVPEQEYTKFSYEFETTE
KDSSNIEITLIGSGTTYLDNLSITELNSTPEILDEPEVKIPTDQEIIDAH
KIYSSDLNFNPSTGNAYINGMYFAPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile C00002493 (RT019)
(residues 617-876)
(SEQ ID NO: 37)
PTYFTNFDDYNNYPSTWSNVNTTNKDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTPEILDEPEVKIPTDQEIIDAH
KIYFADLNFNPSTGNTYINGMYFAPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile CCUG20309 (RT036)
(residues 617-876)
(SEQ ID NO: 38)
PTYFTNFDDYNNYPSTWSNVNTTNKDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTPEILDEPEVKIPTDQEIIDAH
KIYFADLNFNPSTGNTYINGMYFAPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile R20291 (RT027)
(residues 617-876)
(SEQ ID NO: 39)
PTYFTNFDDYNNYPSTWSNVNTTNQDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTPEILDEPEVKIPTDQEIMDAH
KIYFADLNFNPSTGNTYINGMYFAPTQTNKEALDYIQKYRVEATLQYSGF
KDIGTKDKEMRNYLGDPNQPKTNYVNLRSYFTGGENIMTYKKLRIYAITP
DDRELLVLSVD
RBD+2 C. difficile C00009694 (RT244)
(residues 617-876)
(SEQ ID NO: 40)
PTYFTNFDDYNNYPSTWSNVNTTNQDGLQGSANKLNGETKIKIPMSELKP
YKRYVFSGYSKDPLTSNSIIVKIKAKEEKTDYLVPEQGYTKFSYEFETTE
KDSSNIEITLIGSGTTYLDNLSITELNSTPEILDEPEVKIPTDQEIMDAH
KIYFADLNFNPSTGNTYINGMYFAPTQTNKEALDYIQKYRVEATLQYSGF
KDIGTKDKEMRNYLGDPNQPKTNYVNLRSYFTGGENIMTYKKLRIYAITP
DDRELLVLSVD

Mouse Immunization and Subsequent Challenge with CDTa/CDTb

Female C57/BL6 mice were housed under the same conditions. Food, water, bedding, and cages were autoclaved. Mice (n=5) were immunized 3 times at 14-day intervals via intraperitoneal injection (i.p.) route with 10 μg of purified RBD1, RBD2 or RBD1+2 in phosphate-buffered saline (PBS) along with alum as an adjuvant for each injection³⁶. Control mice (n=5) received PBS with alum. Sera were collected. Fourteen days after the third immunization, immunized or control mice were i.p. challenged with a lethal dosage of CDT (CDTa 260 ng/activated CDTb 2870 ng, CDTa/activated CDTb=1:7, n=5 for each protein group) or activated CDTb (2870 ng/mouse, n=5 for each protein group) only, monitored for survival and disease symptoms for 72 hrs.

### ELISA for Anti-RBD1, RBD2 or RBD1+2 IgG, Anti-CDTb IgG/A

ELISA assays were performed as previously described³⁷. Briefly, Costar 96-well ELISA plates were coated with 100 ul/well of RBD1, RBD2, RBD1+2 or CDTb (0.5 ug/ml) at 4° C. overnight. Following washing of the unbound material, plates were blocked with 300 μl of blocking buffer (PBS+5% dry milk) at room temperature for 2 hours. After washing, 100 μl of 10-fold diluted sera were added into each well of the plates and incubated for 1.5 hours at room temperature. Following washing with PBS, 100 μl of mouse IgG-HRP (1:3000) or IgA-HRP were added to each well and incubated for 30 min to 1 hour. Subsequent to a washing step with PBS, substrate TMB was added to allow color development at room temperature for 5-30 min. The reaction was stopped by addition of H₂SO₄ to each well, and the OD values at 450 nm were recorded by a spectrophotometer. Anti-RBD1, RBD2, RBD1+2 or CDTb IgG/A titers of a given sample (serum sample from immunized mice) was defined as the dilution factor at which the OD₄₅₀ₙₘ is greater or equal to 2-fold that of serum sample from non-immunized mice.

### Cell Culture and Intoxication Experiments

Mouse intestinal epithelial CT26 cells were used to assess intoxication experiments. Cells were cultivated in culture dishes (48-well format) and treated with respective toxin components (indicated in figure legends). After 2 hours, images of cells were taken using microscopy BZ-X800 (Keyence Company, IL, USA). Percentage of rounded cells was determined from images.

Hamster Immunization and Hamster Model of C. difficile Infection

Golden Syrian male/female (1:1) hamsters used here were housed individually in cages under the same conditions. Hamsters were orally immunized with either 10 μg or 30 μg RBD2 with alum an adjuvant via intramuscular route for 3 times at 14-days intervals. Control hamsters were immunized with same volume of PBS. Sera were collected and anti-RBD2 IgA or IgG titers were determined by ELISA. Two weeks after the third immunization, hamsters were i.p. administered with one dose of clindamycin at 30 mg/kg followed by oral challenge with 2×10⁴ C. difficile DSM101085 spores 3 days later. The hamsters were monitored for 7 days for diarrhea and other disease symptoms. Tissues (cecum, colon, heart, lung, kidney, brain, spleen, and liver) were collected from non-immunized and non-infected hamster, non-immunized and C. difficile-infected hamster, or RBD2-immunized and C. difficile-infected hamster before death or day 7.

### Statistical Analysis

Animal survivals were analyzed by Kaplan-Meier survival analysis with a log-rank test of significance. When comparing results for two groups, student's unpaired f-test was used for statistical significance. Results are expressed as means±standard errors of means. Differences were considered statistically significant if p<0.05 (*). All statistical analyses were performed using GraphPad Prism software.

The following non-limiting examples illustrate exemplary systems and components thereof in accordance with various embodiments of the disclosure. The examples are merely illustrative and are not intended to limit the disclosure in any way.

### Example 1—Method of Preventing C. difficile Infection (Prophetic)

A 29-year-old female patient is administered a therapeutically effective amount of a vaccine comprised of a protein having SEQ ID NO: 21 and a pharmaceutically acceptable carrier via parenteral injection. The patient is exposed to C. difficile and does not contract CDI.

A 39-year-old male patient is nasally administered a therapeutically effective amount of a vaccine comprised of a protein having SEQ ID NO: 22 and a pharmaceutically acceptable carrier. The patient is exposed to C. difficile and does not contract CDI.

A 45-year-old female patient is administered a therapeutically effective amount of a vaccine comprised of a protein having SEQ ID NO: 23 and a pharmaceutically acceptable carrier and an aluminum salt adjuvant via parenteral injection. The patient is exposed to C. difficile and does not contract CDI.

A 50-year-old female patient is administered a therapeutically effective amount of a vaccine comprised of a protein having SEQ ID NO: 24 and a pharmaceutically acceptable carrier via parenteral injection. The patient is exposed to C. difficile and does not contract CDI.

A 55-year-old female patient is nasally administered a therapeutically effective amount of a vaccine comprised of a protein having SEQ ID NO: 25 and a pharmaceutically acceptable carrier. The patient is exposed to C. difficile and does not contract CDI.

A 30-year-old male patient is administered a therapeutically effective amount of a vaccine comprised of a protein having SEQ ID NO: 26 and a pharmaceutically acceptable carrier and an aluminum salt adjuvant via parenteral injection. The patient is exposed to C. difficile and does not contract CDI.

A 60-year-old male patient is administered a therapeutically effective amount of a vaccine comprised of a protein having SEQ ID NO: 27 and a pharmaceutically acceptable carrier via parenteral injection. The patient is exposed to C. difficile and does not contract CDI.

A 40-year-old female patient is administered a therapeutically effective amount of a vaccine comprised of a protein having SEQ ID NO: 28 and a pharmaceutically acceptable carrier via parenteral injection. The patient is exposed to C. difficile and does not contract CDI.

A 65-year-old male patient is nasally administered a therapeutically effective amount of a vaccine comprised of a protein having SEQ ID NO: 29 and a pharmaceutically acceptable carrier. The patient is exposed to C. difficile and does not contract CDI.

A 41-year-old male patient is administered a therapeutically effective amount of a vaccine comprised of a protein having SEQ ID NO: 30 and a pharmaceutically acceptable carrier and an aluminum salt adjuvant via parenteral injection. The patient is exposed to C. difficile and does not contract CDI.

A 53-year-old male patient is administered a therapeutically effective amount of a vaccine comprised of a protein having SEQ ID NO: 31 and a pharmaceutically acceptable carrier via parenteral injection. The patient is exposed to C. difficile and does not contract CDI.

A 28-year-old male patient is nasally administered a therapeutically effective amount of a vaccine comprised of a protein having SEQ ID NO: 32 and a pharmaceutically acceptable carrier. The patient is exposed to C. difficile and does not contract CDI.

A 42-year-old female patient is administered a therapeutically effective amount of a vaccine comprised of a protein having SEQ ID NO: 33 and a pharmaceutically acceptable carrier via parenteral injection. The patient is exposed to C. difficile and does not contract CDI.

A 44-year-old female patient is nasally administered a therapeutically effective amount of a vaccine comprised of a protein having SEQ ID NO: 34 and a pharmaceutically acceptable carrier. The patient is exposed to C. difficile and does not contract CDI.

A 35-year-old male patient is administered a therapeutically effective amount of a vaccine comprised of a protein having SEQ ID NO: 35 and a pharmaceutically acceptable carrier and an aluminum salt adjuvant via parenteral injection. The patient is exposed to C. difficile and does not contract CDI.

A 48-year-old female patient is administered a therapeutically effective amount of a vaccine comprised of a protein having SEQ ID NO: 36 and a pharmaceutically acceptable carrier via parenteral injection. The patient is exposed to C. difficile and does not contract CDI.

A 55-year-old male patient is administered a therapeutically effective amount of a vaccine comprised of a protein having SEQ ID NO: 37 and a pharmaceutically acceptable carrier via parenteral injection. The patient is exposed to C. difficile and does not contract CDI.

A 60-year-old female patient is administered a therapeutically effective amount of a vaccine comprised of a protein having SEQ ID NO: 38 and a pharmaceutically acceptable carrier via parenteral injection. The patient is exposed to C. difficile and does not contract CDI.

A 58-year-old male patient is administered a therapeutically effective amount of a vaccine comprised of a protein having SEQ ID NO: 39 and a pharmaceutically acceptable carrier via parenteral injection. The patient is exposed to C. difficile and does not contract CDI.

A 48-year-old male patient is administered a therapeutically effective amount of a vaccine comprised of a protein having SEQ ID NO: 40 and a pharmaceutically acceptable carrier via parenteral injection. The patient is exposed to C. difficile and does not contract CDI.

A 35-year-old female patient is administered a therapeutically effective amount of anti-RBD2 serum via parenteral injection. The patient is exposed to C. difficile and does not contract CDI.

A 55-year-old male patient is administered a therapeutically effective amount of anti-RBD1+2 serum via parenteral injection. The patient is exposed to C. difficile and does not contract CDI.

### Example 2—Method of Treating C. difficile Infection (Prophetic)

A 49-year-old male patient presents with severe diarrhea and weight loss. A diagnosis of CDI is confirmed. The patient is administered a therapeutically effective amount of a therapeutic agent comprised of anti-RBD2 serum via parenteral injection. After a period of time, the patient's symptoms resolve. The patient is subsequently tested and C. difficile is not found.

A 59-year-old female patient presents with severe diarrhea and weight loss. A diagnosis of CDI is confirmed. The patient is rectally administered a therapeutically effective amount of a therapeutic agent comprised of anti-RBD1+2 serum. After a period of time, the patient's symptoms resolve. The patient is subsequently tested and C. difficile is not found.

A 35-year-old male patient presents with diarrhea and weight loss. A diagnosis of CDI is confirmed. The patient is administered a therapeutically effective amount of a therapeutic agent comprised of a protein having SEQ ID NO: 21 and a pharmaceutically acceptable carrier and an aluminum salt adjuvant via parenteral injection. After a period of time, the patient's symptoms resolve. The patient is subsequently tested and C. difficile is not found.

A 30-year-old male patient presents with diarrhea and weight loss. A diagnosis of CDI is confirmed. The patient is administered a therapeutically effective amount of a therapeutic agent comprised of a protein having SEQ ID NO: 22 and a pharmaceutically acceptable carrier via parenteral injection. After a period of time, the patient's symptoms resolve. The patient is subsequently tested and C. difficile is not found.

A 45-year-old female patient presents with diarrhea and weight loss. A diagnosis of CDI is confirmed. The patient is administered a therapeutically effective amount of a therapeutic agent comprised of a protein having SEQ ID NO: 23 and a pharmaceutically acceptable carrier via parenteral injection. After a period of time, the patient's symptoms resolve. The patient is subsequently tested and C. difficile is not found.

A 45-year-old male patient presents with diarrhea and weight loss. A diagnosis of CDI is confirmed. The patient is nasally administered a therapeutically effective amount of a therapeutic agent comprised of a protein having SEQ ID NO: 24 and a pharmaceutically acceptable carrier. After a period of time, the patient's symptoms resolve. The patient is subsequently tested and C. difficile is not found.

A 38-year-old female patient presents with diarrhea and weight loss. A diagnosis of CDI is confirmed. The patient is administered a therapeutically effective amount of a therapeutic agent comprised of a protein having SEQ ID NO: 25 and a pharmaceutically acceptable carrier via parenteral injection. After a period of time, the patient's symptoms resolve. The patient is subsequently tested and C. difficile is not found.

A 43-year-old male patient presents with diarrhea and weight loss. A diagnosis of CDI is confirmed. The patient is administered a therapeutically effective amount of a therapeutic agent comprised of a protein having SEQ ID NO: 26 and a pharmaceutically acceptable carrier and an aluminum salt adjuvant via parenteral injection. After a period of time, the patient's symptoms resolve. The patient is subsequently tested and C. difficile is not found.

A 48-year-old female patient presents with diarrhea and weight loss. A diagnosis of CDI is confirmed. The patient is nasally administered a therapeutically effective amount of a therapeutic agent comprised of a protein having SEQ ID NO: 27 and a pharmaceutically acceptable carrier. After a period of time, the patient's symptoms resolve. The patient is subsequently tested and C. difficile is not found.

A 49-year-old female patient presents with diarrhea and weight loss. A diagnosis of CDI is confirmed. The patient is administered a therapeutically effective amount of a therapeutic agent comprised of a protein having SEQ ID NO: 28 and a pharmaceutically acceptable carrier via parenteral injection. After a period of time, the patient's symptoms resolve. The patient is subsequently tested and C. difficile is not found.

A 53-year-old female patient presents with diarrhea and weight loss. A diagnosis of CDI is confirmed. The patient is administered a therapeutically effective amount of a therapeutic agent comprised of a protein having SEQ ID NO: 29 and a pharmaceutically acceptable carrier and an aluminum salt adjuvant via parenteral injection. After a period of time, the patient's symptoms resolve. The patient is subsequently tested and C. difficile is not found.

A 30-year-old male patient presents with diarrhea and weight loss. A diagnosis of CDI is confirmed. The patient is administered a therapeutically effective amount of a therapeutic agent comprised of a protein having SEQ ID NO: 30 and a pharmaceutically acceptable carrier via parenteral injection. After a period of time, the patient's symptoms resolve. The patient is subsequently tested and C. difficile is not found.

A 56-year-old male patient presents with diarrhea and weight loss. A diagnosis of CDI is confirmed. The patient is nasally administered a therapeutically effective amount of a therapeutic agent comprised of a protein having SEQ ID NO: 31 and a pharmaceutically acceptable carrier. After a period of time, the patient's symptoms resolve. The patient is subsequently tested and C. difficile is not found.

A 62-year-old male patient presents with diarrhea and weight loss. A diagnosis of CDI is confirmed. The patient is administered a therapeutically effective amount of a therapeutic agent comprised of a protein having SEQ ID NO: 32 and a pharmaceutically acceptable carrier via parenteral injection. After a period of time, the patient's symptoms resolve. The patient is subsequently tested and C. difficile is not found.

A 51-year-old female patient presents with diarrhea and weight loss. A diagnosis of CDI is confirmed. The patient is administered a therapeutically effective amount of a therapeutic agent comprised of a protein having SEQ ID NO: 33 and a pharmaceutically acceptable carrier and an aluminum salt adjuvant via parenteral injection. After a period of time, the patient's symptoms resolve. The patient is subsequently tested and C. difficile is not found.

A 44-year-old male patient presents with diarrhea and weight loss. A diagnosis of CDI is confirmed. The patient is administered a therapeutically effective amount of a therapeutic agent comprised of a protein having SEQ ID NO: 34 and a pharmaceutically acceptable carrier via parenteral injection. After a period of time, the patient's symptoms resolve. The patient is subsequently tested and C. difficile is not found.

A 41-year-old female patient presents with diarrhea and weight loss. A diagnosis of CDI is confirmed. The patient is administered a therapeutically effective amount of a therapeutic agent comprised of a protein having SEQ ID NO: 35 and a pharmaceutically acceptable carrier and an aluminum salt adjuvant via parenteral injection. After a period of time, the patient's symptoms resolve. The patient is subsequently tested and C. difficile is not found.

A 61-year-old female patient presents with diarrhea and weight loss. A diagnosis of CDI is confirmed. The patient is nasally administered a therapeutically effective amount of a therapeutic agent comprised of a protein having SEQ ID NO: 36 and a pharmaceutically acceptable carrier. After a period of time, the patient's symptoms resolve. The patient is subsequently tested and C. difficile is not found.

A 44-year-old male patient presents with diarrhea and weight loss. A diagnosis of CDI is confirmed. The patient is administered a therapeutically effective amount of a therapeutic agent comprised of a protein having SEQ ID NO: 37 and a pharmaceutically acceptable carrier via parenteral injection. After a period of time, the patient's symptoms resolve. The patient is subsequently tested and C. difficile is not found.

A 36-year-old female patient presents with diarrhea and weight loss. A diagnosis of CDI is confirmed. The patient is administered a therapeutically effective amount of a therapeutic agent comprised of a protein having SEQ ID NO: 38 and a pharmaceutically acceptable carrier via parenteral injection. After a period of time, the patient's symptoms resolve. The patient is subsequently tested and C. difficile is not found.

A 54-year-old male patient presents with diarrhea and weight loss. A diagnosis of CDI is confirmed. The patient is administered a therapeutically effective amount of a therapeutic agent comprised of a protein having SEQ ID NO: 39 and a pharmaceutically acceptable carrier via parenteral injection. After a period of time, the patient's symptoms resolve. The patient is subsequently tested and C. difficile is not found.

### CONCLUSION

The inventors constructed a vaccine containing RBD2 which exhibited potent efficacy as a new vaccine candidate in experimental mouse models of CDI. The data showed that, not only does the RBD2 protein and the RBD1+2 protein represent an effective vaccine candidate, but also anti-RBD2 and anti-RBD1+2 serum may represent an alternative therapy against CDI.

### REFERENCES

1. Leffler, D. A.; Lamont, J. T., Clostridium difficile Infection. New England Journal of Medicine 2015, 372, 1539-1548.
2. Kuehne, S. A.; Cartman, S. T.; Heap, J. T.; Kelly, M. L.; Cockayne, A.; Minton, N. P., The role of toxin A and toxin B in Clostridium difficile infection. Nature 2010, 467, 711-U97.
3. Giesemann, T.; Egerer, M.; Jank, T.; Aktories, K., Processing of Clostridium difficile toxins. J Med Microbiol 2008, 57, 690-696.
4. Perelle, S.; Gibert, M.; Bourlioux, P.; Corthier, G.; Popoff, M. R., Production of a complete binary toxin (actin-specific ADP-ribosyltransferase) by Clostridium difficile CD196. Infect Immun 1997, 65, 1402-7.
5. Cook, M. E.; Varney, K. M.; Godoy-Ruiz, R.; Weber, D. J., (1)H(N), (13)C, and (15)N resonance assignments of the Clostridioides difficile receptor binding domain 2 (CDTb, residues 757-876). Biomol NMR Assign 2021, 15, 35-39.
6. Hung, Y. P.; Huang, I. H.; Lin, H. J.; Tsai, B. Y.; Liu, H. C.; Liu, H. C.; Lee, J. C.; Wu, Y. H.; Tsai, P. J.; Ko, W. C., Predominance of Clostridium difficile Ribotypes 017 and 078 among Toxigenic Clinical Isolates in Southern Taiwan. PLoS One 2016, 11, e0166159.
7. Crobach, M. J. T.; Vernon, J. J.; Loo, V. G.; Kong, L. Y.; Pechine, S.; Wilcox, M. H.; Kuijper, E. J., Understanding Clostridium difficile Colonization. Clin Microbiol Rev 2018, 31.
8. Ernst, K.; Landenberger, M.; Nieland, J.; Norgaard, K.; Frick, M.; Fois, G.; Benz, R.; Barth, H., Characterization and Pharmacological Inhibition of the Pore-Forming Clostridioides difficile CDTb Toxin. Toxins (Basel) 2021, 13.
9. Abeyawardhane, D. L.; Godoy-Ruiz, R.; Adipietro, K. A.; Varney, K. M.; Rustandi, R. R.; Pozharski, E.; Weber, D. J., The Importance of Therapeutically Targeting the Binary Toxin from Clostridioides difficile. Int JMol Sci 2021, 22.
10. Gulke, I.; Pfeifer, G.; Liese, J.; Fritz, M.; Hofmann, F.; Aktories, K.; Barth, H., Characterization of the enzymatic component of the ADP-ribosyltransferase toxin CDTa from Clostridium difficile. Infect Immun 2001, 69, 6004-11.
11. Xie, J.; Horton, M.; Zorman, J.; Antonello, J. M.; Zhang, Y.; Arnold, B. A.; Secore, S.; Xoconostle, R.; Miezeiewski, M.; Wang, S.; Price, C. E.; Thiriot, D.; Goerke, A.; Gentile, M. P.; Skinner, J. M.; Heinrichs, J. H., Development and optimization of a high-throughput assay to measure neutralizing antibodies against Clostridium difficile binary toxin. Clin Vaccine Immunol 2014, 21, 689-97.
12. Barth, H.; Aktories, K.; Popoff, M. R.; Stiles, B. G., Binary bacterial toxins: biochemistry, biology, and applications of common Clostridium and Bacillus proteins. Microbiol Mol Biol Rev 2004, 68, 373-402, table of contents.
13. Xu, X.; Godoy-Ruiz, R.; Adipietro, K. A.; Peralta, C.; Ben-Hail, D.; Varney, K. M.; Cook, M. E.; Roth, B. M.; Wilder, P. T.; Cleveland, T.; Grishaev, A.; Neu, H. M.; Michel, S. L. J.; Yu, W.; Beckett, D.; Rustandi, R. R.; Lancaster, C.; Loughney, J. W.; Kristopeit, A.; Christanti, S.; Olson, J. W.; MacKerell, A. D.; Georges, A. D.; Pozharski, E.; Weber, D. J., Structure of the cell-binding component of the Clostridium difficile binary toxin reveals a di-heptamer macromolecular assembly. Proc Natl Acad Sci USA 2020, 117, 1049-1058.
14. Landenberger, M.; Nieland, J.; Roeder, M.; Norgaard, K.; Papatheodorou, P.; Ernst, K.; Barth, H., The cytotoxic effect of Clostridioides difficile pore-forming toxin CDTb. Biochim Biophys Acta Biomembr 2021, 1863, 183603.
15. Fischer, S.; Uckert, A. K.; Landenberger, M.; Papatheodorou, P.; Hoffmann-Richter, C.; Mittler, A. K.; Ziener, U.; Hagele, M.; Schwan, C.; Muller, M.; Kleger, A.; Benz, R.; Popoff, M. R.; Aktories, K.; Barth, H., Human peptide alpha-defensin-1 interferes with Clostridioides difficile toxins TcdA, TcdB, and CDT. FASEB J 2020, 34, 6244-6261.
16. Korbmacher, M.; Fischer, S.; Landenberger, M.; Papatheodorou, P.; Aktories, K.; Barth, H., Human alpha-Defensin-5 Efficiently Neutralizes Clostridioides difficile Toxins TcdA, TcdB, and CDT. Front Pharmacol 2020, 11, 1204.
17. Abeyawardhane, D. L.; Godoy-Ruiz, R.; Adipietro, K. A.; Varney, K. M.; Rustandi, R. R.; Pozharski, E.; Weber, D. J., The importance of therapeutically targeting the binary toxin from Clostridioides difficile. International Journal of Molecular Sciences 2021, 22, 2926.
18. Kordus, S. L.; Thomas, A. K.; Lacy, D. B., Clostridioides difficile toxins: mechanisms of action and antitoxin therapeutics. Nat Rev Microbiol 2022, 20, 285-298.
19. Papatheodorou, P.; Carette, J. E.; Bell, G. W.; Schwan, C.; Guttenberg, G.; Brummelkamp, T. R.; Aktories, K., Lipolysis-stimulated lipoprotein receptor (LSR) is the host receptor for the binary toxin Clostridium difficile transferase (CDT). Proc Natl Acad Sci USA 2011, 108, 16422-7.
20. Simpson, M.; Bilverstone, T.; Leslie, J.; Donlan, A.; Uddin, M. J.; Petri, W. A.; Marin, N.; Kuehne, S.; Minton, N. P.; Petri, W. A., Jr., Clostridioides difficile Binary Toxin Binding Component Increases Virulence in a Hamster Model. Open Forum Infect Dis 2023, 10, ofad040.
21. Gerding, D. N.; Johnson, S.; Rupnik, M.; Aktories, K., Clostridium difficile binary toxin CDT: mechanism, epidemiology, and potential clinical importance. Gut microbes 2014, 5, 15-27.
22. Martinez-Melendez, A.; Cruz-López, F.; Morfin-Otero, R.; Maldonado-Garza, H. J.; Garza-González, E., An Update on Clostridioides difficile Binary Toxin. Toxins 2022, 14, 305.
23. Frentrup, M.; Zhou, Z.; Steglich, M.; Meier-Kolthoff, J. P.; Goker, M.; Riedel, T.; Bunk, B.; Spröer, C.; Overmann, J.; Blaschitz, M., A publicly accessible database for genome sequences supports tracing of transmission chains and epidemics. 2020.
24. Riedel, T.; Wetzel, D.; Hofmann, J. D.; Plorin, S. P. E. O.; Dannheim, H.; Berges, M.; Zimmermann, O.; Bunk, B.; Schober, I.; Spröer, C., High metabolic versatility of different toxigenic and non-toxigenic Clostridioides difficile isolates. International Journal of Medical Microbiology 2017, 307, 311-320.
25. Stabler, R. A.; He, M.; Dawson, L.; Martin, M.; Valiente, E.; Corton, C.; Lawley, T. D.; Sebaihia, M.; Quail, M. A.; Rose, G., Comparative genome and phenotypic analysis of Clostridium difficile 027 strains provides insight into the evolution of a hypervirulent bacterium. Genome biology 2009, 10, 1-15.
26. Steglich, M.; Hofmann, J. D.; Helmecke, J.; Sikorski, J.; Spröer, C.; Riedel, T.; Bunk, B.; Overmann, J.; Neumann-Schaal, M.; Nübel, U., Convergent loss of ABC transporter genes from Clostridioides difficile genomes is associated with impaired tyrosine uptake and p-cresol production. Front Microbiol 2018, 9, 901.
27. Grofß, U.; Brzuszkiewicz, E.; Gunka, K.; Starke, J.; Riedel, T.; Bunk, B.; Spröer, C.; Wetzel, D.; Poehlein, A.; Chibani, C., Comparative genome and phenotypic analysis of three Clostridioides difficile strains isolated from a single patient provide insight into multiple infection of C. difficile. BMC genomics 2018, 19, 1-14.
28. Chang, S. Y.; Song, K. P., ADP-ribosylating binary toxin genes of Clostridium difficile strain CCUG 20309. DNA Sequence 2001, 12, 115-120.
29. Fatoba, A. J.; Fatoba, D. O.; Babalola, S. O., Pangenome and subtractive genomic analysis of Clostridioides difficile reveals putative drug targets. Journal of Proteins and Proteomics 2022, 1-10.
30. Kumar, A.; Davenport, K. W.; Vuyisich, G.; Kunde, Y. A.; Johnson, S. L.; Chain, P. S. G.; Dichosa, A. E. K.; Rodriguez-Palacios, A., Complete genome sequences of historic Clostridioides difficile food-dwelling ribotype 078 strains in Canada identical to that of the historic human clinical strain M120 in the United Kingdom. Microbiology Resource Announcements 2018, 7, e00853-18.
31. Kumar, S.; Stecher, G.; Li, M.; Knyaz, C.; Tamura, K., MEGA X: molecular evolutionary genetics analysis across computing platforms. Molecular biology and evolution 2018, 35, 1547.
32. Zimmermann, L.; Stephens, A.; Nam, S.-Z.; Rau, D.; Kübler, J.; Lozajic, M.; Gabler, F.; Söding, J.; Lupas, A. N.; Alva, V. J. J. o. m. b., A completely reimplemented MPI bioinformatics toolkit with a new HHpred server at its core. Journal of molecular biology 2018, 430, 2237-2243.
33. Gabler, F.; Nam, S. Z.; Till, S.; Mirdita, M.; Steinegger, M.; Söding, J.; Lupas, A. N.; Alva, V. J. C. P. i. B., Protein Sequence Analysis Using the MPI Bioinformatics Toolkit. Current Protocols in Bioinformatics 2020, 72, e108.
34. Waterhouse, A. M.; Procter, J. B.; Martin, D. M.; Clamp, M.; Barton, G. J., Jalview Version 2—a multiple sequence alignment editor and analysis workbench. Bioinformatics 2009, 25, 1189-1191.
35. Livingstone, C. D.; Barton, G. J., Protein sequence alignments: a strategy for the hierarchical analysis of residue conservation. Bioinformatics 1993, 9, 745-756.
36. Lee, S.; Picking, W. L.; Tzipori, S., The immune response of two microbial antigens delivered intradermally, sublingually, or the combination thereof Microbes Infect 2014, 16, 796-803.
37. Wang, Y. K.; Yan, Y. X.; Kim, H. B.; Ju, X.; Zhao, S.; Zhang, K.; Tzipori, S.; Sun, X., A chimeric protein comprising the glucosyltransferase and cysteine proteinase domains of toxin B and the receptor binding domain of toxin A induces protective immunity against Clostridium difficile infection in mice and hamsters. Hum Vaccin Immunother 2015, 11, 2215-22.
38. Riedel, T.; Wetzel, D.; Hofmann, J. D.; Plorin, S.; Dannheim, H.; Berges, M.; Zimmermann, O.; Bunk, B.; Schober, I.; Spröer, C.; Liesegang, H.; Jahn, D.; Overmann, J.; Gross, U.; Neumann-Schaal, M., High metabolic versatility of different toxigenic and non-toxigenic Clostridioides difficile isolates. Int J Med Microbiol 2017, 307, 311-320.
39. Stabler, R. A.; He, M.; Dawson, L.; Martin, M.; Valiente, E.; Corton, C.; Lawley, T. D.; Sebaihia, M.; Quail, M. A.; Rose, G.; Gerding, D. N.; Gibert, M.; Popoff, M. R.; Parkhill, J.; Dougan, G.; Wren, B. W., Comparative genome and phenotypic analysis of Clostridium difficile 027 strains provides insight into the evolution of a hypervirulent bacterium. Genome Biol 2009, 10, R102.
40. Steglich, M.; Hofmann, J. D.; Helmecke, J.; Sikorski, J.; Spröer, C.; Riedel, T.; Bunk, B.; Overmann, J.; Neumann-Schaal, M.; Nubel, U., Convergent Loss of ABC Transporter Genes From Clostridioides difficile Genomes Is Associated With Impaired Tyrosine Uptake and p-Cresol Production. Front Microbiol 2018, 9, 901.
41. Gross, U.; Brzuszkiewicz, E.; Gunka, K.; Starke, J.; Riedel, T.; Bunk, B.; Spröer, C.; Wetzel, D.; Poehlein, A.; Chibani, C.; Bohne, W.; Overmann, J.; Zimmermann, O.; Daniel, R.; Liesegang, H., Comparative genome and phenotypic analysis of three Clostridioides difficile strains isolated from a single patient provide insight into multiple infection of C. difficile. BMC Genomics 2018, 19, 1.
42. Chang, S. Y.; Song, K. P., ADP-ribosylating binary toxin genes of Clostridium difficile strain CCUG 20309. DNA Seq 2001, 12, 115-20.
43. Abiodun J. Fatoba, D. O. F. S. O. B., Pangenome and subtractive genomic analysis of Clostridioides difficile reveals putative drug targets. Journal of Proteins and Proteomics volume 2022, pages 247-256.
44. Kumar, A.; Davenport, K. W.; Vuyisich, G.; Kunde, Y. A.; Johnson, S. L.; Chain, P. S. G.; Dichosa, A. E. K.; Rodriguez-Palacios, A., Complete Genome Sequences of Historic Clostridioides difficile Food-Dwelling Ribotype 078 Strains in Canada Identical to That of the Historic Human Clinical Strain M120 in the United Kingdom. Microbiol Resour Announc 2018, 7.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A Clostridioides difficile (C. difficile) vaccine comprising:
a therapeutically effective amount of a protein or fragment thereof from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2).

2. The vaccine of claim 1, wherein the RBD2 has a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

3. The vaccine of claim 1, wherein the RBD1+2 has a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

4. The vaccine of claim 1, further comprising a pharmaceutically acceptable carrier.

5. The vaccine of claim 4, further comprising an adjuvant.

6. The vaccine of claim 5, wherein the adjuvant is an aluminum salt.

7. A method of preventing a C. difficile infection (CDI) in a patient in need thereof comprising:
administering to the patient a therapeutically effective amount of a therapeutic agent, the therapeutic agent comprising
a serum containing antibodies to receptor binding domain 2 (RBD2) protein from binary toxin (CDT) of C. difficile;
a serum containing antibodies to receptor binding domain 1 (RBD1) protein and RBD2 protein (RBD1+2) from the binary toxin (CDT) of C. difficile; or
a vaccine, the vaccine comprising
a therapeutically effective amount of a protein or fragment thereof from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2); and
a pharmaceutically acceptable carrier;
wherein the administration of the therapeutically effective amount of the therapeutic agent to the patient prevents CDI.

8. The method of claim 7, wherein the therapeutic agent is the serum wherein the serum is anti-RBD2 serum.

9. The method of claim 7, wherein the therapeutic agent is the vaccine.

10. The method of claim 9, wherein the vaccine is comprised of the RBD2 having a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 80% homology thereto.

11. The method of claim 9, wherein the vaccine is comprised of the RBD1+2 having a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 80% homology thereto.

12. The method of claim 7, wherein the vaccine further comprises an adjuvant.

13. The method of claim 12, wherein the adjuvant is an aluminum salt.

14. A method of treating a C. difficile infection (CDI) in a patient in need thereof comprising:
administering to the patient a therapeutically effective amount of a therapeutic agent, the therapeutic agent comprising
a serum containing antibodies to receptor binding domain 2 (RBD2) protein from binary toxin (CDT) of C. difficile;
a serum containing antibodies to receptor binding domain 1 (RBD1) protein and RBD2 protein (RBD1+2) from the binary toxin (CDT) of C. difficile; or
a vaccine, the vaccine comprising
a therapeutically effective amount of a protein or fragment thereof from component CDTb of binary toxin (CDT) of C. difficile wherein the protein or fragment thereof is receptor binding domain 2 (RBD2) protein or a protein comprising receptor binding domain 1 (RBD1) protein and RBD2 protein (RBD1+2); and
a pharmaceutically acceptable carrier;
wherein the administration of the therapeutically effective amount of the therapeutic agent to the patient prevents CDI.

15. The method of claim 14, wherein the therapeutic agent is the serum wherein the serum is anti-RBD2 serum.

16. The method of claim 14, wherein the therapeutic agent is the vaccine.

17. The method of claim 16, wherein the therapeutic agent is the vaccine comprised of the RBD2 having a sequence of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, a cDNA encoding SEQ ID NO: 21, a cDNA encoding SEQ ID NO: 22, a cDNA encoding SEQ ID NO: 23, a cDNA encoding SEQ ID NO: 24, a cDNA encoding SEQ ID NO: 25, a cDNA encoding SEQ ID NO: 26, a cDNA encoding SEQ ID NO: 27, a cDNA encoding SEQ ID NO: 28, a cDNA encoding SEQ ID NO: 29, a cDNA encoding SEQ ID NO: 30 or a sequence having at least 90% homology thereto.

18. The method of claim 16, wherein the therapeutic agent is the vaccine comprised of the RBD1+2 having a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a cDNA encoding SEQ ID NO: 31, a cDNA encoding SEQ ID NO: 32, a cDNA encoding SEQ ID NO: 33, a cDNA encoding SEQ ID NO: 34, a cDNA encoding SEQ ID NO: 35, a cDNA encoding SEQ ID NO: 36, a cDNA encoding SEQ ID NO: 37, a cDNA encoding SEQ ID NO: 38, a cDNA encoding SEQ ID NO: 39, a cDNA encoding SEQ ID NO: 40, or a sequence having at least 90% homology thereto.

19. The method of claim 16, further comprising an adjuvant.

20. The method of claim 19, wherein the adjuvant is an aluminum salt.
